(19) United States
(12) Patent Application Publication
(10) Pub. No.: US 2024/0029893 A1
(43) Pub. Date: Jan. 25, 2024

(54) SYSTEMS AND METHODS FOR ASSESSING AND MITIGATING PERSONAL HEALTH HAZARDS IN AN INDOOR ENVIRONMENT FOR A PLURALITY OF OCCUPANTS

(71) Applicant: Building4Health Inc., Keswick, VA (US)

(72) Inventors: Stephanie Hughes Taylor, Stowe, VT (US); Michael Bennett Solovay, New York, NY (US); Peter Railey Taylor, SR., Keswick, VA (US)

(21) Appl. No.: 18/352,631

(22) Filed: Jul. 14, 2023

Related U.S. Application Data
(60) Provisional application No. 63/389,603, filed on Jul. 15, 2022.

(51) Int. Cl. G16H 50/30; G01N 33/00; G16H 15/00

(52) U.S. Cl. CPC G16H 50/30; G01N 33/0075; G16H 15/00

(57) ABSTRACT

A system and a method include receiving from environmental sensors, environmental output data measurements. The environmental sensors are located at a site location. A health impact scoring algorithm computes a plurality of health impact scores from the environmental output data measurements. An overall health impact score at the site location is computed from the health impact score having a lowest value. A machine learning model generates a personal recommendation for reducing a health impact from a verified environmental hazard type to each occupant in the site location. At least one of: the overall health impact score, the at least one verified environmental hazard type, the occupant exposure time, or the personal recommendation are displayed on a computing device. An instruction is sent to environment-controlling equipment located at the site location to change an operational parameter of the environment-controlling equipment to mitigate the at least one verified environmental hazard type.

### FIELD OF TECHNOLOGY

The present disclosure generally relates to systems and methods for assessing and mitigating personal health hazards in an indoor environment for a plurality of occupants.

### BACKGROUND OF TECHNOLOGY

A computer network platform/system may include a group of computers (e.g., clients, servers) and other computing hardware devices that are linked together through one or more communication channels to facilitate communication and/or resource-sharing, via one or more specifically programmed graphical user interfaces (GUIs) of the present disclosure, among a wide range of users.

### SUMMARY OF DESCRIBED SUBJECT MATTER

In some embodiments, the present disclosure provides an exemplary technically improved computer-based method that includes at least the following steps of continuously receiving, by a processor, over a communication network, from a plurality of environmental sensors, a plurality of environmental output data measurements of a plurality of environmental parameters, where the plurality of environmental parameters may include:
(i) an ozone parameter,
(ii) a humidity parameter,
(iii) a temperature parameter,
(iv) a carbon dioxide parameter,
(v) a carbon monoxide parameter,
(vi) a nitrous dioxide parameter,
(vii) a sulfur dioxide parameter,
(viii) a total volatile organic compound (tVOC) parameter, and
(ix) a particulate matter parameter;

The plurality of environmental sensors may be located inside of a site location, outside of the site location, or both. Each particular sensor of the plurality of environmental sensors may be configured to continuously output a particular environmental output data measurement for a particular environmental parameter of the plurality of environmental parameters to obtain the plurality of environmental output data measurements. A particular health impact score for each particular environmental parameter of the plurality of environmental parameters may be automatically determined, by the processor, based on the particular environmental output data measurement for the particular environmental parameter and at least one other environmental output data measurement from the plurality of environmental output data measurements to obtain a plurality of health impact scores associated with the plurality of environmental parameters. Each health impact score of the plurality of health impact scores may be associated with a particular health impact on at least one occupant in the site location. An overall health impact score may be defined, by the processor, based on a lowest health impact score from the plurality of health impact scores. A personalized health data for each occupant from the plurality of occupants at the site location may be received by the processor. The processor may determine: at least one verified environmental hazard type associated with at least one environmental parameter from the plurality of environmental parameters when at least one health impact score from the plurality of health impact scores is less than at least one predefined threshold score that is unique to the at least one environmental parameter, and at least one exposure time that the at least one verified environmental hazard type has been present at the site location. At least one machine learning model may be utilized, by the processor, to generate at least one personalized recommendation for reducing a health impact from the at least one verified environmental hazard type to each occupant based on inputting the plurality of health impact scores and the personalized health data for each occupant into the at least one machine learning model. At least one first instruction may be transmitted, over the communication network, by the processor, to display on a computing device at least one of:
(i) the overall health impact score,
(ii) the at least one verified environmental hazard type,
(iii) the at least one exposure time for the at least one verified environmental hazard type,
(iv) the at least one personalized recommendation, or
(v) at least one possible health effect on each occupant based on the at least one exposure time to the at least one verified environmental hazard type; and
   At least one second instruction may be automatically transmitted, by the processor, over the communication network, to at least one environment-controlling equipment associated with the site location to change an operational parameter of the at least one environment-controlling equipment to mitigate the at least one verified environmental hazard type.

In some embodiments, the present disclosure provides an exemplary technically improved computer-based system that includes at least a memory and a processor. The processor may be configured to execute computer code stored in the memory that may cause the processor to continuously receive over a communication network, from a plurality of environmental sensors, a plurality of environmental output data measurements of a plurality of environmental parameters. The plurality of environmental parameters may include:
(i) an ozone parameter,
(ii) a humidity parameter,
(iii) a temperature parameter,
(iv) a carbon dioxide parameter,
(v) a carbon monoxide parameter,
(vi) a nitrous dioxide parameter,
(vii) a sulfur dioxide parameter,
(viii) a total volatile organic compound (tVOC) parameter, and
(ix) a particulate matter parameter;
   The plurality of environmental sensors may be located inside of a site location, outside of the site location, or both. Each particular sensor of the plurality of environmental sensors may be configured to continuously output a particular environmental output data measurement for a particular environmental parameter of the plurality of environmental parameters to obtain a plurality of environmental output data measurements. A particular health impact score may be automatically determined for each particular environmental parameter of the plurality of environmental parameters based on the particular environmental output data measurement for the particular environmental parameter and at least one other environmental output data measurement from the plurality of environmental output data measurements to obtain a plurality of health impact scores associated with the plurality of environmental parameters. Each health impact score of the plurality of health impact scores may be associated with a particular health impact on at least one occupant in the site location. An overall health impact score may be defined based on a lowest health impact score from the plurality of health impact scores. A personalized health data for each occupant from the plurality of occupants at the site location may be received. The processor may be configured to determine at least one verified environmental hazard type associated with at least one environmental parameter from the plurality of environmental parameters when at least one health impact score from the plurality of health impact scores is less than at least one predefined threshold score that is unique to the at least one environmental parameter, and at least one exposure time that the at least one verified environmental hazard type has been present at the site location. At least one machine learning model may be utilized to generate at least one personalized recommendation for reducing a health impact from the at least one verified environmental hazard type to each occupant based on inputting the plurality of health impact scores and the personalized health data for each occupant into the at least one machine learning model. At least one first instruction may be transmitted over the communication network, to display on a computing device at least one of:
(i) the overall health impact score,
(ii) the at least one verified environmental hazard type,
(iii) the at least one exposure time for the at least one verified environmental hazard type,
(iv) the at least one personalized recommendation, or
(v) at least one possible health effect on each occupant based on the at least one exposure time to the at least one verified environmental hazard type; and
   At least one second instruction may be automatically transmitted over the communication network, to at least one environment-controlling equipment associated with the site location to change an operational parameter of the at least one environment-controlling equipment to mitigate the at least one verified environmental hazard type.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a diagram of a system for assessing and mitigating personal health hazards in an indoor environment at a site location for a plurality of occupants in accordance with one or more embodiments of the present disclosure;

FIG. 2 schematically illustrates an SB computing device at an exemplary site location in accordance with one or more embodiments of the present disclosure;

FIG. 3 is a diagram of an exemplary SB computing device in accordance with one or more embodiments of the present disclosure;

FIG. 4 is an exemplary snapshot of the graphic user interface (GUI) displayed on a display in accordance with one or more embodiments of the present disclosure;

FIG. 5 is a first flow diagram using a plurality of environmental sensors in a health impact score algorithm in accordance with one or more embodiments of the present disclosure;

FIG. 6 is a table showing the breakpoint values for different pollutants in accordance with one or more embodiments of the present disclosure;

FIG. 7 is a table showing a second embodiment for computing health index scores in accordance with one or more embodiments of the present disclosure;

FIG. 8 is a table showing human health impact of measured indoor factors in accordance with one or more embodiments of the present disclosure;

FIG. 9 illustrates a flowchart of an exemplary method for assessing and managing a health impact of an indoor environment at a site location in accordance with one or more embodiments of the present disclosure;

FIG. 10 is a diagram showing various metrics used for formulating an indoor air quality (IAQ) human health standard in accordance with one or more embodiments of the present disclosure;

FIG. 11 is a second flow diagram showing an algorithm for computing an overall health impact score in accordance with one or more embodiments of the present disclosure;

FIG. 12 is a third flow diagram showing an algorithm for computing a set of individual health impact scores in accordance with one or more embodiments of the present disclosure;

FIG. 13 is a diagram illustrating quantifiable health effects of primary and interacting indoor pollutants as well as thermal conditions in accordance with one or more embodiments of the present disclosure; and

FIG. 14 illustrates a flowchart of an exemplary method for assessing and mitigating personal health hazards in an indoor environment at a site location for a plurality of occupants in accordance with one or more embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

Embodiments of the present disclosure describe a system using a device for assessing and managing a health impact of an indoor environment at a site location. The indoor environment at the site location in which at least one person occupies may have an impact on the health of the at least one person. The computer-based systems and methods disclosed herein may optimize a plurality of environmental factors at the site location so as to ensure safe and healthy indoor environments for its occupants. For example, the system and methods described herein may be used to optimize indoor environments. The optimized indoor environments may not only reduce the probability of infectious aerosol transmission but may also promote a robust immune system and mitigate health problems, such as for example, fatigue, decreased cognitive functioning, stress, anxiety and depression, allergies and autoimmune disorders, dermatitis, bronchospasm and reactive airway disease (asthma), respiratory inflammation, eye irritation and excessive tearing.

In some embodiments, the system may be configured to receive location data for each of a plurality of occupants at the site location and/or personalized medical data for each of the plurality of occupants. Once the device assesses a presence of at least one health hazard in an indoor environment at a site location as well as the time that the at least one health hazard is present in the indoor environment of the site location, the system may determine personalized exposure data for each of the plurality of occupants and may generate personalized health alerts for each of the plurality of occupants exposed to the at least one health hazard.

The term site location may refer to at least one room or indoor environment in a house, building, and/or buildings at one or more locations that may be monitored by a central monitoring facility, as well as the outdoor area proximal to the at least one room or indoor environment at the site location.

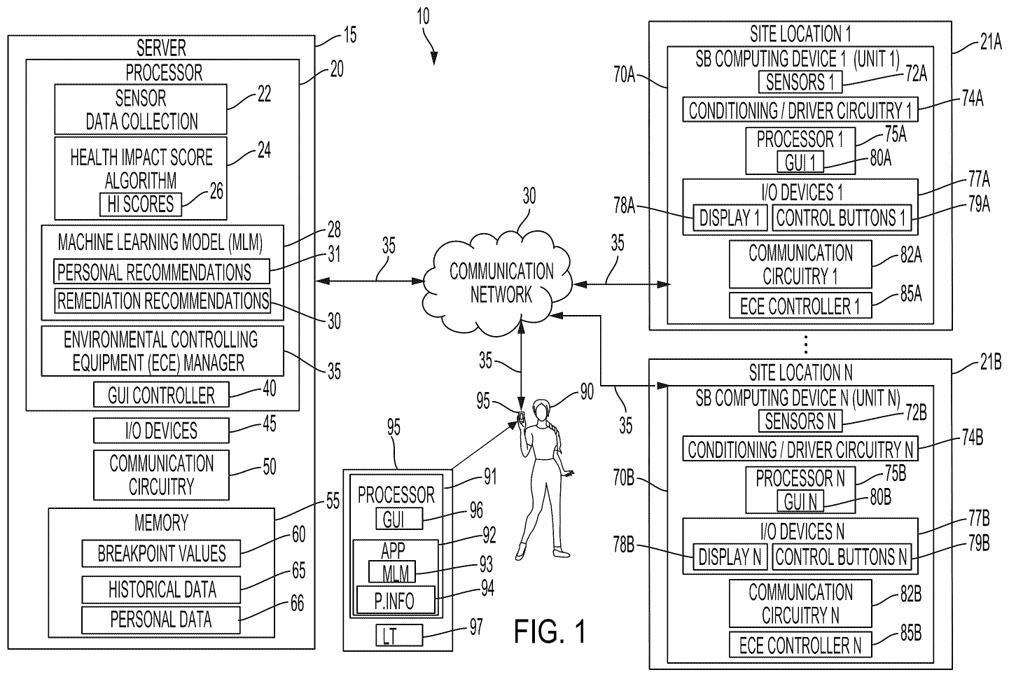

FIG. 1 is a diagram of a system 10 for assessing and mitigating personal health hazards in an indoor environment at a site location for a plurality of occupants in accordance with one or more embodiments of the present disclosure. The system 10 may include a server 15, a plurality of N sensor-based (SB) computing devices 70A and 70B denoted UNIT 1 and UNIT N, where N is an integer at a respective plurality of N site locations 21A and 21B, and a mobile computing device 95 with a graphic user interface (GUI) 96 on a display associated with a user 90 that all communicate 35 over a communication network 30. The mobile computing device associated with the user may also be a computer, tablet, laptop, for example with a GUI 96. Note that FIG. 1 illustrates, merely for conceptual clarity and not by way of limitation, one sensor-based computing device at one site location, but there may be any suitable number of sensor-based computing devices at a single site location.

In some embodiments, the server 15 may include a processor 20, a memory 55, input and output (I/O) devices 45 and a communication circuitry 50 for enabling the server 15 to communicate 35 over the communication network 30. The processor 20 may execute software modules to enable the functions of the system 10 as described herein. The software modules may include a sensor data collection 22 module for collecting sensor data from each of the N sensor-based computing devices over the communication network 30, a health impact score algorithm 24 for computing health impact (HI) scores 26 for the collected sensor data, a machine learning model 28 trained to output remediation recommendations 30 to improve the indoor environment of the site location and/or personal recommendations 31 to reduce the resultant damage from detected health hazards in the indoor environment for at least one occupant in the indoor environment, an environmental controlling equipment (ECE) manager 35 for sending instructions over the communication network for controlling environment controlling equipment at the site location, and a GUI controller 40 for sending instructions to display, for example, health impact scores, remediation recommendations, and/or any other suitable metrics on any of the graphic user interfaces (GUIs) used in the system 10.

In some embodiments, the memory 55 may store a breakpoint value 60 database, where the breakpoint values may be used to compute the health impact scores and/or a historical data 65 database may be used to store any suitable parameters needed to display to a user, the historical health impact scores over any desired time interval. The memory 55 may store any suitable database for storing data for performing the functions described herein such as a personal database with personal information related to.

In some embodiments, at the site location 1 21A, a sensor-based (SB) computing device1 70A may be located. The SB computing device1 70A may include a plurality of sensors 72A denoted SENSORS 1, conditioning and sensor data driver circuitry 1 74A for conditioning the sensor data by converting the analog output data from the sensors to digital sensor data via analog-to-digital converters, input and output (I/O) devices1 77A, such as a display 1 78A, a microphone and/or speaker (not shown), and/or control buttons 1 79A, for example, a processor 1 75A that may be configured to display a GUI 1 80A on the display 1 78A, a communication circuitry 1 82A for enabling the SB computing device to communicate 35 over the communication network 30, and/or an environmental controlling equipment controller 1 85A circuitry for controlling equipment at the site location for optimizing the environment in which the SB computing device1 82A is located. The circuitry of the SB computing device1 70A may be powered by a battery, a solar cell, and/or via a connection a mains electricity such as via a wall outlet, for example. The control buttons 1 79A may be used for powering UNIT1 on and off, changing any readouts on the display1 78A, or used for any suitable purpose.

In some embodiments, similarly at the Nth site location 21B, a sensor-based (SB) computing deviceN 70B may be located. The SB computing deviceN 70B may include a plurality of sensors 72B denoted SENSORS N, conditioning and sensor data driver circuitry N 74B for conditioning the sensor data by converting the analog output data from the sensors to digital sensor data via ana log-to-digital converters, input and output (I/O) devicesN 77B, such as a displayN 78B, a microphone and/or speaker (not shown), and/or control buttons N 79B, for example, a processorN 75B that may be configured to display a GUI N 80B on the display N 78B, a communication circuitry N 82B for enabling the SB computing device to communicate 35 over the communication network 30, and/or an environmental controlling equipment controller N 85B circuitry for controlling equipment at the site location for optimizing the environment in which the SB computing device N 70B is located. The circuitry of the SB computing deviceN 70B may be powered by a battery, a solar cell, and/or via a connection a mains electricity such as via a wall outlet, for example. The control buttons N 79B may be used for powering UNITN on and off, changing any readouts on the displayN 78B, or used for any suitable purpose.

It should be noted that the Nth SB computing deviceN 70B and its elements may be referred to hereinafter as SB computing device 70 at the site location 20, the processor 75 controlling a GUI 80, etc.

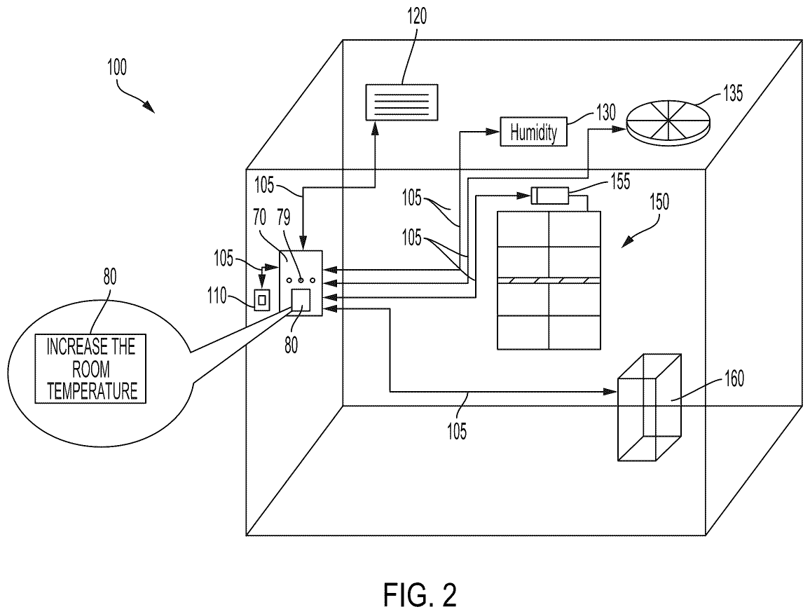

FIG. 2 schematically illustrates the sensors-based computing device 70 at an exemplary site location 100 in accordance with one or more embodiments of the present disclosure. The exemplary site location 100 may be a room (e.g., an indoor environment) in a building with computer-controlled environment control equipment. The sensor-based computing device 70 may communicate 35 over the communication network 30 with the server 30. The SB computing device may relay sensor data (e.g., typically digitized sensor data) received from the plurality of sensors over the communication network to the sensor data collection 22 module in the server 15. Subsequently, the SB computing device 70 may receive a first instruction from the GUI controller over the communication network 30 to display recommendations (e.g., “INCREASE THE ROOM TEMPERATURE”) on the GUI 80 and/or to activate an audible alert such as, for example, when an environmental hazard type in the room is detected and verified. Similarly, the SB computing device 70 may receive a second instruction from the ECE manager 35 over the communication network 30 to change an operational parameter environment control equipment located at the site location so as to mitigate the environmental hazard type in the room.

In some embodiments, the site location 100 may include the computer-controlled environment control equipment such as, for example, a computer-controlled thermostat 110, a computer-controlled vent 120 to vent air between the interior to the room to the outside, a computer-controlled humidifier/dehumidifier unit 130, a computer-controlled ceiling fan for dispersing indoor air, a window 150 that may be opened or closed by a computer-controlled motorized window actuator 155, and/or a computer-controlled air filter 160 in the room. Each of the computer-controlled environment control equipment may have circuitry to allow each of the computer-controlled environment control equipment to communicate 105 with the sensor-based computing device 70. In other embodiments, any or all of the computer-controlled environment control equipment may be operated manually, for example, by the user 90 receiving a remediation recommendation to “open the window”, for example, either on GUI 80 and/or GUI 96.

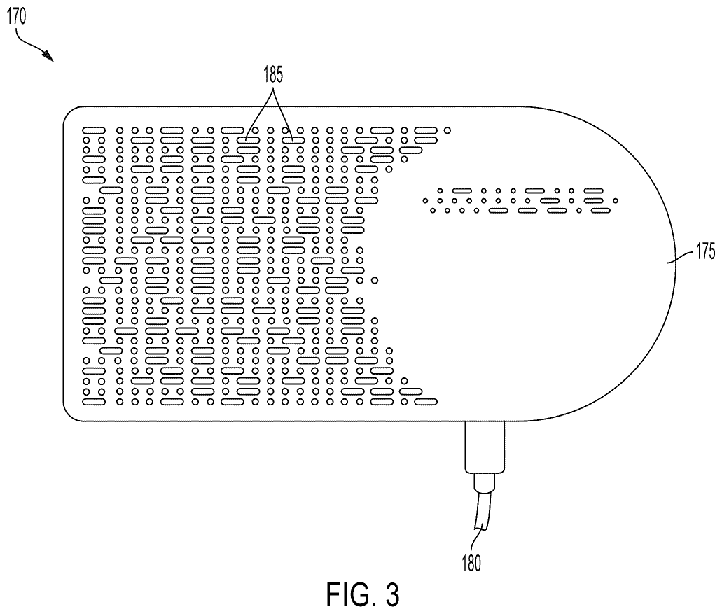

The embodiments shown in FIGS. 1 and 2 are merely for visual and conceptual clarity and not by way of the embodiments disclosed herein. The SB computing device 70 may include only sensors, processing and communication circuitry without a display such that the SB computing device 70 may merely sense the environment at the site location using the plurality of sensors and may relay the sensor data to the server 15 as shown in the example of FIG. 3 below. Subsequently, the server 15 may issue alerts to a user 90 via the GUI 96 on the mobile computing device associated with a user 90, for example. The user 90 may be an occupant of the room. The alert may be an audible alarm, a visual alert, an electronic alert message, or any combination thereof.

FIG. 3 is a diagram of an exemplary SB computing device 170 in accordance with one or more embodiments of the present disclosure. The SB computing device 170 may include circuitry deployed within a case 175 formed from any suitable material. The circuitry may include the plurality of sensors 72, the conditioning and driver circuitry 74, the processor 75 and the communication circuitry 82 for communicating 35 over the communication network 30. A power cord 180 may be connected to wall output for example, to power the circuitry. The case 175 may include a plurality of holes 185 formed in the case 175 for allowing each of the plurality of sensors 72 to contact and/or sample the environment proximal to the SB computing device 170.

In some embodiments, the SB computing device 170 and/or the SB computing device 70 may communicate 35 over a Wireless Fidelity (Wi-Fi) network, or any suitable communication protocol. The sensor data from each SB computing device may be captured and then transmitted to a cloud environment such as Amazon Web Services (AWS), for example.

In some embodiments, the server 15 (e.g., the sensor data collection 22 module) may use an application programming interface (API) and data fetcher that reaches into the TSI cloud (e.g., cloud computing services from TSI healthcare) and pulls the data need by the health impact score algorithm 24, which is subsequently loaded to the cloud environment (e.g., AWS). The TSI cloud may continuously receive the data from the sensors, but the sensor data collection 22 module may pull the needed data to the AWS cloud at predefined time intervals such as every 15 minutes. Thus, the environmental output data measurements may be received from a third-party database.

In some embodiments, the SB computing device 170 as shown in FIG. 3 may have dimensions of 6-7 inches long and 5 inches wide with a thickness of 1 inch. The plurality of sensors 72 in the SB computing device 170 may sense environment hazards at a site location (e.g., a room) covering an area ranging from 1000-5000 square feet.

In some embodiments, once a particular SB computing device 170 may detect at least one verified environment health hazard type when the processor 20 analyzes the sensor data from the plurality of sensors in the particular SB computing device 170 located in a particular room, the server 15 may issue alerts to the GUI 96 of the computing devices associated with the users that are the occupants of the particular room.

In some embodiments, the mobile computing device 95 may include a processor 91 that is configured to control the graphic user interface (GUI) 96 on a display associated with the user 90, location tracking (LT) circuitry 97 such as operating on global positioning system (GPS), WiFi or any suitable location tracking for identifying a location of the user 90, for example, in the site location. The processor 91 may execute computer code stored in a memory of the mobile computing device 95 such as a personal health management software application 92 that may include a trained machine learning model (MLM) module 93 and personal medical information (P.INFO) 94 software module of the user.

In some embodiments, the processor 91 communicating 35 over the communication network 30 may receive data from any of the N sensors-based computing devices denoted 70A . . . 70B in any of the N site locations 21A . . . 21B. When the user 90 enters any of the plurality of site locations 21A . . . 21B whose SB computing devices 70A . . . 70B is communicatively coupled to the server 15, the LT 97 may transmit a location of the user 90 such as a particular indoor space within the site location. The processor 91 of the mobile computing device 91 of the user 90 and/or the processor 20 of the server 15 may track the location of the user 90 and determine the total amount of time that the user is located within a particular indoor space. Thus, if one SB computing device of the SB computing devices 70A . . . 70B detects at least one environmental health hazard, the processor 91 of the user computing device 91 and/or the processor 20 of the server 15 may determine the exposure time of the user 90 to the at least one health hazard at the location of the one SB computing device.

In some embodiments, the mobile computing device 91 may be a smartphone, a wearable device, a tablet, and/or a laptop with appropriate location tracking (LT) circuitry 97 and communication circuitry for communicating 35 with the server 15 and/or any of the N sensors-based computing devices denoted 70A . . . 70B in any of the N site locations 21A . . . 21B.

In some embodiments, the plurality of sensors 72 may be located in at least one smartphone and/or at least one wearable device associated with at least one occupant (e.g., the user 90). In other embodiments, some sensors from the plurality of sensors 72 may be located in the indoor environment at the site location and other sensors from the plurality of sensors 72 may be deployed in the at least one smartphone and/or the at least one wearable device associated with the at least one occupant in the indoor environment.

In some embodiments, the server 15 and/or the mobile computing device 91 associated with the user 90 may be configured to access and download from a medical provider database, personal medical information of the user.

In some embodiments, the server 15 may send batch alerts to the GUI 96 on a plurality of computing devices 95 associated a plurality of users where each of the plurality of users may be at least one site location with at least one verified environmental hazard type. For example, the server 15 may track the details of each user that have entered the building and their location in the building using the location tracking (LT) 97, for example, in the user's computing device 95.

In some embodiments, a user computing device 95 may be a central facility computing device of a business or company. A plurality of SB computing devices 70 may be deployed in a plurality of site locations 20 (e.g., rooms) in a plurality of buildings at different geographical locations associated with the business. The plurality of SB computing devices 70 may relay the sensor data in each room in each building to the central facility computing device associated with the business or company. The GUI 96 may display the status of the environment and may alert a maintenance officer of the business or company about the presence of any verified health hazard types in any of the rooms.

In some embodiments, the company may be, for example, a very large nursing home company that has hundreds of nursing homes, and/or extended care facilities. The user such as a maintenance officer may see a home screen on GUI 96 that may show a map of the United States with click buttons in each location around the country where a particular nursing home and/or extended care facility may be located. The maintenance officer in a central facility in New York, for example, may be able to access a page for a nursing home in Orlando, Florida, for example, and further drill down to a particular room in the Orlando nursing home to view the health statistics that are sensed by a particular SB computing device from the plurality of SB computing devices 70 in the particular room.

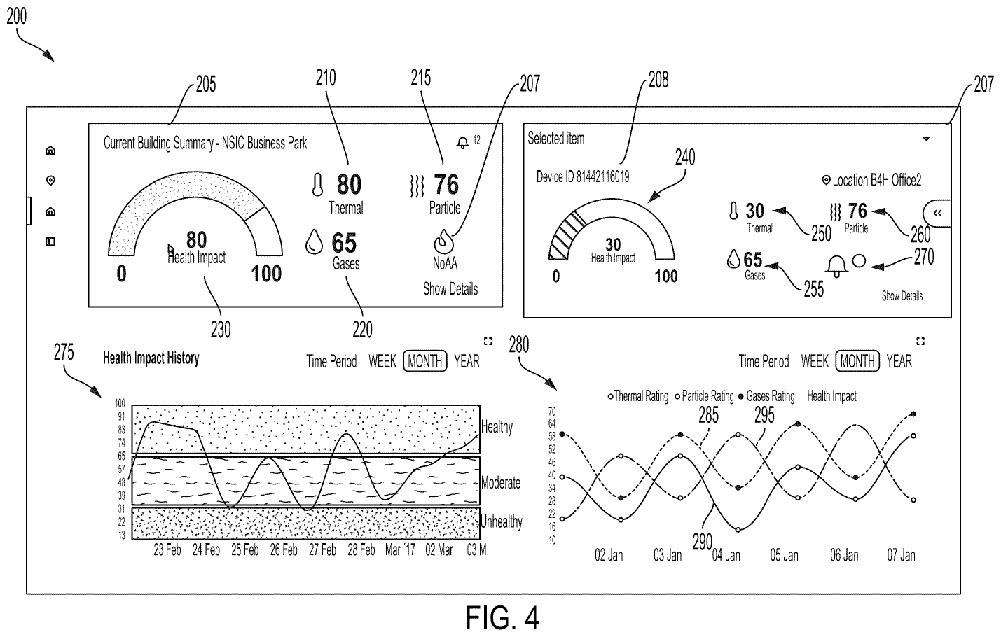

FIG. 4 is an exemplary snapshot 200 of the graphic user interface (GUI) 96 displayed on the display 95 in accordance with one or more embodiments of the present disclosure. The exemplary snapshot 200 in this example may refer to a building with a plurality of rooms. The SB computing device 170 such as shown in FIG. 3 may be deployed in each room. The computing device 95 associated with the user 90, such as the building manager or handyman maintaining the building, may receive environment health sensor updates on the overall building 205 and in a particular room 207. The GUI 96 may include an overall health impact rating (HIR) 230 in the building (e.g., NSIC Business Park in this exemplary case), a thermal HIR 210 based on the temperature detected in the rooms of building environment, a gases HIR 220 based on gases detected in the rooms of the building environment, and a particle HIR 215 based on particulate matter or particles detected in the air in the rooms of the building environment.

In some embodiments, the health impact ratings detected from the SB computing device 170 for a particular room 207 (e,g, Location—Office Room 2 in the NSIC Business Park Building) may be identified by a device identification number 208 (e.g., 81442116019 in this exemplary case). The GUI 96 on unit in the particular room 207 shows that the overall HIR 240 for this room is 30, the thermal HIR 250 for this room is 30, the gases HIR 255 for this room is 65 and the particle HIR 60 for this room is 76.

In some embodiments, the HIR may indicate a level of environmental hazard of a particular type such as thermal, gases, and/or particles (e.g., particulate matter in the air). The HIR rating may include four health impact quartiles, for example, in a particular environment where an HIR of 75-100 is Excellent, an HIR of 50-75 is Good to Acceptable, an HIR of 25-50 Needs Remediation, and an HIR of 0-25 is Hazardous. In other embodiments, the HIR ranges may be defined as 0-24 as being unhealthy, 25-49 as being moderate, and 50-100 as healthy. Any suitable range definition may be used.

In some embodiments, predefined thresholds for the overall, particle, gases and particle HIR may be defined for example at a value of 49, for example. Thus the SB computing device 170 may issue an alert 270 due to detecting an unhealthy temperature HIR rating of 30 in the particular room 207 (e,g, Location—Office Room 2 in the NSIC Business Park Building).

In some embodiments, the GUI 96 may display an overall health impact rating history 275 of the entire building or a particular room over a time period of a week, a month, and/or a year that the user may choose on the GUI 96. Similarly, the GUI 96 may display a historical thermal HIR 295, a historical gases HIR 285, and a historical particle HIR 290.

In some embodiment, the GUI 96 may activate an icon 207 indicating that the outdoor air quality outside of the building is good based on measurements from the external sensors. In other embodiments, the SB computing device 170 unit may be placed outside of the building to compare the air quality inside of site location (e.g., inside the building) to the air quality outside of the site location (e.g., outside of the building).

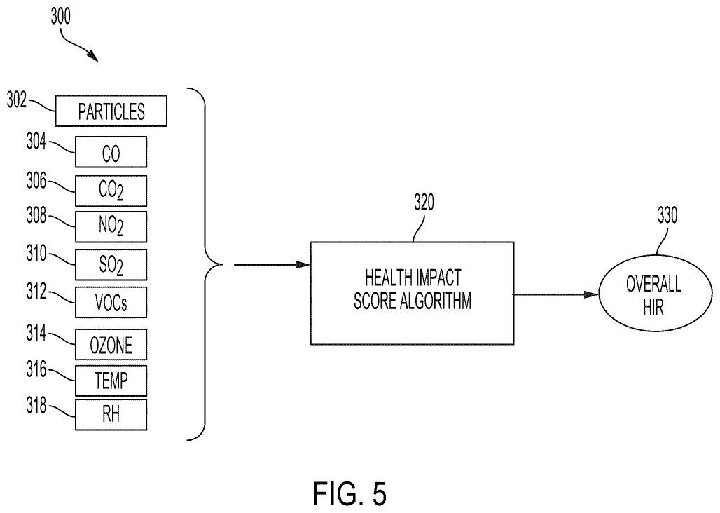

FIG. 5 is a first flow diagram 300 using a plurality of health sensors in a health impact score algorithm 320 in accordance with one or more embodiments of the present disclosure. The plurality of health sensors 72 may include an ozone sensor, outputting an ozone output data measurement 314, a humidity sensor, outputting a humidity or a relative humidity RH output data measurement 318, a temperature sensor, outputting a temperature output data measurement (TEMP) 316, a carbon dioxide sensor, outputting a carbon dioxide (CO2) output data measurement 306, a carbon monoxide sensor, outputting a carbon monoxide (CO) output data measurement 304, a nitrous dioxide sensor, outputting a nitrous dioxide (NO2) output data measurement 308, a sulfur dioxide sensor, outputting a sulfur dioxide (SO2) output data measurement 310, a total volatile organic compound (tVOC) sensor, outputting a VOCs output data measurement 312, and at least one particulate matter sensor, outputting at least one particulate matter output data measurement (PARTICLES) 302.

Note that the ten unique environmental sensor types for collecting the ten sensor data values have been chosen based on the individual and interacting impact of measured components on human physiology and microbial viability. The sensor data from each sensor type may be weighted according to its influence on other indoor pollutants. If sensor data from a particular sensor type is not collected due to sensor malfunction, for example, the algorithms may become less comprehensive. Conversely, if a sensor detects a low insignificant level of a given pollutant, the integrity of the algorithm may still be maintained. Note that the use of ten unique environmental sensor types is not by way of limitation of the embodiments disclosed herein, any suitable number of unique environmental sensor types may be used.

The sensors may include (1) Airthings Wave Plus sensors, Airthings, Wergelandsveien 7, 0167 Oslo, Norway, (2) Kaiterra Sensedge Commercial Air Quality Monitor, Kaiterra, Crans Montana, VS3974, Switzerland, and (3) TSI AirAssure sensor, TSI Inc., 500 Cardigan Road, Shoreview, Minnesota 55126 U.S.A.

In some embodiments, the total volatile organic compound (tVOC) sensor may be configured to detect specific volatile organic compounds in the air such as phenol, toluene, trichlorobenzenes, trichloroethylene, vinyl chloride, and/or formaldehyde, for example.

In some embodiments. the at least one particulate matter sensor may include a 10 μm PM₁₀ particulate matter sensor, a 2.5 μm PM_{2.5} particulate matter sensor, and a 1 μm PK particulate matter sensor. Thus the at least one particulate matter output data measurement (PARTICLES) 302 may be based on a PM_{2.5} particulate matter output data measurement, a PM_{2.5} particulate matter output data measurement, and a PM₁ particulate matter output data measurement.

Each of the plurality of output data measurements from each of the respective sensors may be inputted into an health impact score algorithm 320 (or the health impact score algorithm 24 of FIG. 1). Each of the health impact scores for each pollutant may be outputted from the health impact score algorithm 320. In some embodiments, the HIR values may be calculated when normalizing the HI(f) values to an ozone and relative humidity factor based on the ozone output data measurement 314, and the relative humidity RH output data measurement 318.

In some embodiments, the health impact score HI(RH) related to the relative humidity sensor output data measurement 318 may be computed from equation (1) where C_{RH} is the relative humidity concentration obtained from the relative humidity sensor output data measurement 318, breakpoint values BP_{Hi} and BP_{Lo} of C_{RH} and index breakpoint values I_{Hi} of BP_{Hi} and I_{Lo} of BP_{Lo}, constants that may be obtained from medical studies and/or exposure studies. These breakpoint values may be stored in the breakpoint value database 60.

$$HI ( RH ) = \frac{( {C}_{RH} - {BP}_{LO} )}{( {BP}_{Hi} - {BP}_{Lo} )} x ( {I}_{Hi} - {I}_{Lo} ) + {I}_{Lo} \qquad ( 1 )$$

Similarly, the health impact score HI(O3) related to the ozone sensor output data measurement 318 may be computed from equation (2) where C_{O3} is the ozone concentration obtained from the ozone sensor output data measurement 314, RH_{f} is the relative humidity factor based on Equation (1), breakpoint values BP_{Hi} and BP_{Lo} of C_{O3} and index breakpoint values I_{Hi} of BP_{Hi} and I_{Lo} of BP_{Lo}, constants that may be obtained from medical studies and/or exposure studies. These breakpoint values may be stored in the breakpoint value database 60.

$$HI ( {O}_{3} ) = \frac{( {C}_{O 3} X {RH}_{f} - {BP}_{LO} )}{( {BP}_{Hi} - {BP}_{Lo} )} x ( {I}_{Hi} - {I}_{Lo} ) + {I}_{Lo} \qquad ( 2 )$$

Finally, the health impact ratings HI(f) may be computed using Equation (3) for each of the other f pollutants may be based on the pollutant concentration C_{f}, the relative humidity factor RH_{f}, and an ozone factor O3_{f}. The other f pollutants may include particulate matter PARTICLES, carbon monoxide CO, carbon dioxide CO₂, nitrous dioxide NO₂, sulfur dioxide SO₂, total volatile organic compound tVOCs, and temperature TEMP each having respective concentrations denoted by C_{f}={C_{PM10}, C_{PM2.5}, C_{PM1}, C_{tVOC}, C_{NO2}, C_{SO2}, C_{CO}, C_{CO2}, C_{T}}

$$HI ( f ) = \frac{( {C}_{f} X {RH}_{i} X O {3}_{f} - {BP}_{LO} )}{( {BP}_{Hi} - {BP}_{Lo} )} x ( {I}_{Hi} - {I}_{Lo} ) + {I}_{Lo} \qquad ( 3 )$$

Thus, the health impact score algorithm may compute using Equations (1)-(3), the set of health index score (HI) values including a 10 μm particulate matter health index score HI(PM₁₀), a 2.5 μm particulate matter health index score HI(PM_{2.5}), a 1 μm particulate matter health index score HI(PM₁), a total volatile organic compound health index score HI(tVOC), a nitrous dioxide health index score HI(NO₂), a sulfur dioxide health index score HI(SO₂), a carbon monoxide health index score HI(CO), an ozone health index score HI(O₃), a carbon dioxide health index score HI(CO₂), a relative humidity health index score HI(RH), a temperature health index score HI(Temp)}. Each of the health index scores is based is least in part on the environmental output data measurements from the plurality of environmental sensors.

As shown in FIG. 4, the HIR for gases 220 may be the lowest HI value related to gases in the set of HI values (e.g., HI(tVOC), HI(NO₂), HI(SO₂), HI(CO), HI(O₃), HI(CO₂)), the HIR for particles 215 may be the lowest HI value related to particles (e.g., HI(PM₁₀), HI(PM_{2.5}), HI(PM₁)) in the set of HI values, the HIR for thermal conditions 210 is HI(Temp). The overall HIR 330 may be derived from the lowest value in the set of HI values, e.g., the lowest of any one of {HI(PM₁₀), HI(PM_{2.5}), HI(PM₁), HI(tVOC), HI(NO₂), HI(SO₂), HI(CO), HI(O3), HI(CO₂), HI(RH), HI(Temp)}. In some embodiments, HIR=100−{normalized maximum of HI(PM₁₀, PM_{2.5}, PM₁, tVOC, NO₂, SO₂, CO, O₂, CO₂, RH, TEMP)}.

In some embodiments, the health impact score algorithm 320 may use short term acceptable exposure levels making the limits conservative and safest for each health impact zone. Continuous monitoring may be used to capture all conditions where each of the HI values in the set above is weighted according to interactions with concurrent relative humidity and ozone to establish a single, dynamic, multipollutant aggregated health impact rating. As a result, pollutant concentrations may be related to the safest international medical thresholds.

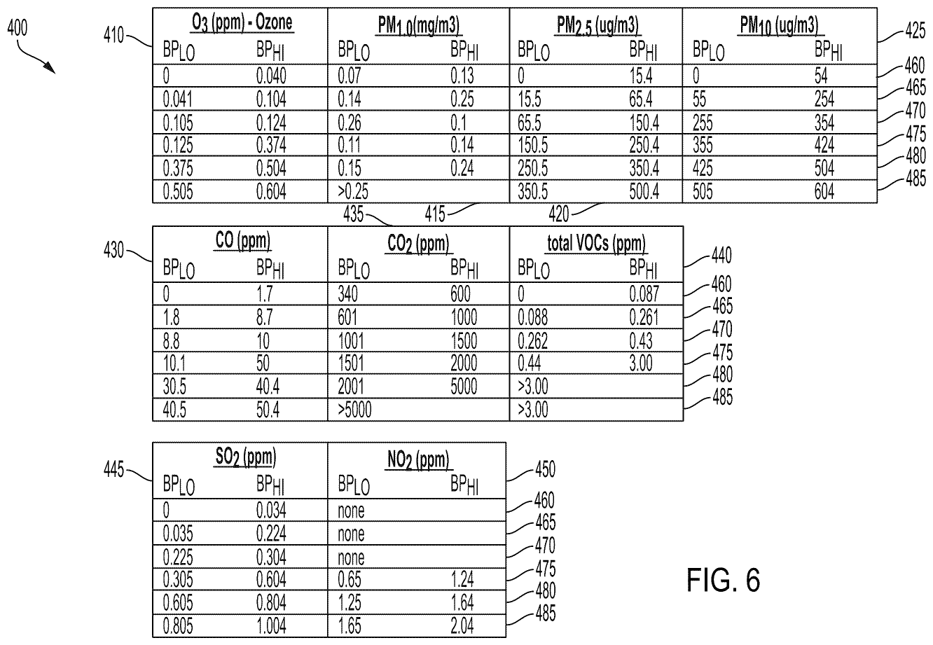

FIG. 6 is a table 400 showing the breakpoint values for different pollutants in accordance with one or more embodiments of the present disclosure. The aggregate HI values may be determined by selecting the pollutant with the highest pollutant index so as to avoid exaggerated measurements (ambiguity) found with adding HI values or lack of action (eclipsing) found with averaging HI values. Thus, the overall HIR is computed using the lowest HI value at a particular site location and not by averaging, adding, or multiplying individual pollutant indices. The HI values may be used to create a continuous scale which can be categorized as good 460, normal 465, unhealthy for sensitive groups 470, unhealthy 475, very unhealthy 480, and hazardous 485) which are used in the computation of the HI(f) values. The health impact score (HI) values may be normalized to create a health impact rating (HIR).

In the table 400, the breakpoint values BP_{LO} and BP_{HI} are for ozone 410, for PM_{1.0} 415, for PM_{2.5} 420, for PM₁₀ 425, for CO 430, for CO₂ 435, for total VOCs 440, for SO₂ 445, and for NO₂ 450. Each shows breakpoint values in each of the six zones.

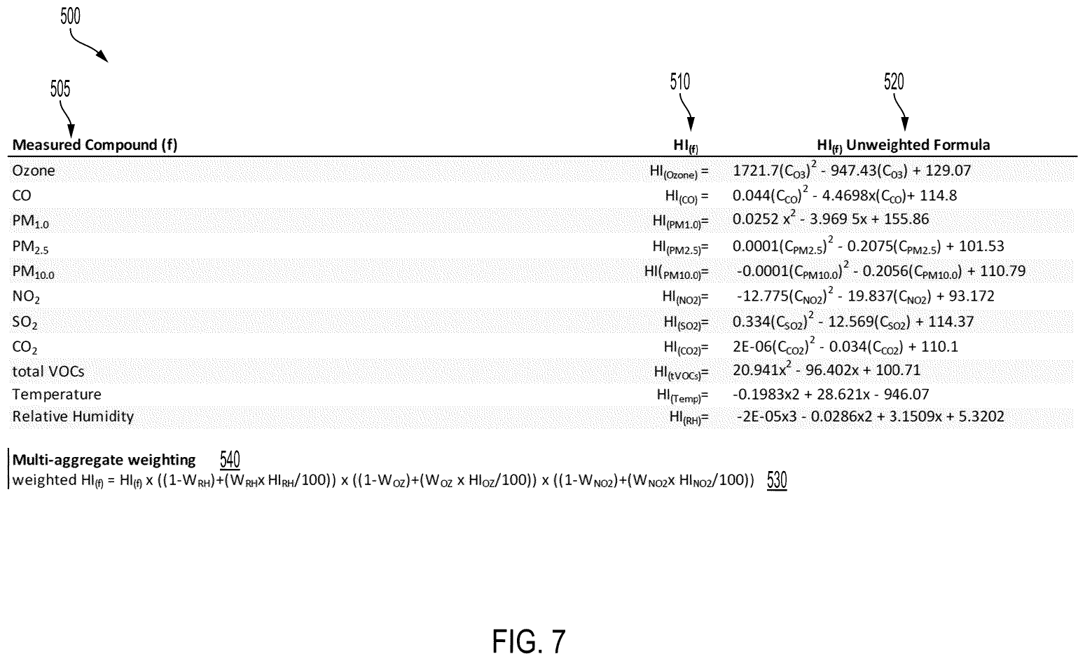

FIG. 7 is a table 500 showing a second embodiment for computing health impact scores in accordance with one or more embodiments of the present disclosure. The equations shown for computing the HI(f) scores are a different, second embodiment for computing HI(f) in contrast to Equations (1)-(3). In this exemplary second embodiment, the HI scores may be measured having maximum exposure levels taken over a predefined time interval such as eight hours, for example. There is no need to consider the six threshold regions as shown in FIG. 6 for use in the first HI(f) model. In this case, the HI(f) model may use a continuous slope model that may be fit to the HI score data using two fitting polynomials. Thus, each pollutant, or measured compound (f) 505 in table 500 for each HI(f) 510 may have a unique equation 520 as shown in FIG. 7 in contrast to Equation (3) in the first HI(f) model. Each HI(f) unweighted formula 520 may be a function of the pollutant concentration value and/or the square and/or cube of the pollutant concentration value. In some of the equations 520, the pollutant concentration may be generically denoted as x, x 2, etc. So for tVOC, for example, x in the HI(tvoc) equation for tVOC in FIG. 7 is the same as C_{tvoc} in Equation (3) above, and so forth. Multi-aggregate weighting 540 may be applied to the unweighted HI(f) parameter. The weights may be taken from the peer reviewed medical and epidemiological literature including the Cochran database, a public database for healthcare decision-making, to adjust the HI(f) values. A weighting equation 530 may be used to weight each computed HI (t) value by relative humidity, ozone, and/or NO₂. In some embodiments, these weights may be chosen to account for the effects of one or a combination of each pollutant on the health impact score for any given environment.

In some embodiments, each of the health index scores HI described in Equations (1)-(3) and/or in FIG. 7 is based is least in part on the environmental output data measurements from the plurality of environmental sensors. In other embodiments, environmental output data measurements may be the measured pollutant concentrations (e.g., C_{RH}, C_{O3}, C_{PM10}, C_{PM2.5}, C_{PM1}, C_{tVOC}, C_{NO2}, C_{SO2}, C_{CO}, C_{CO2}, C_{T}).

It should be noted that the two exemplary health impact algorithms for computing the health impact scores as shown in FIGS. 5-7 and defined in equations (1)-(3) are not by way of limitation of the embodiments disclosed herein. Any suitable health impact algorithm may be used by system 10 and/or the SB computing device N 70B to compute the health impact scores as used herein to assess environmental hazards and for automatically managing the indoor environment for optimum occupant health.

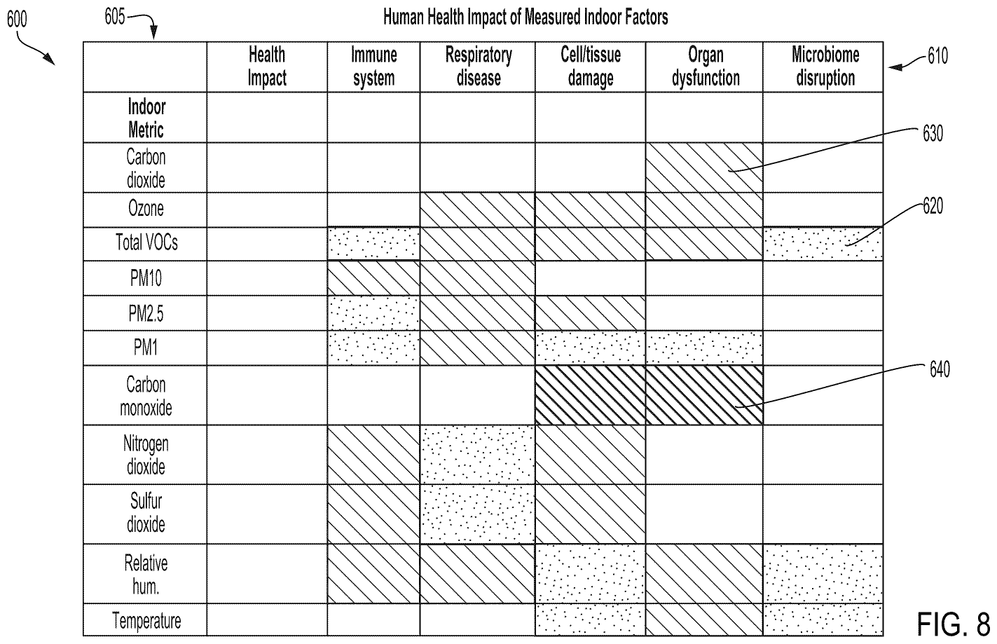

FIG. 8 is a table 600 showing human health impact of measured indoor factors in accordance with one or more embodiments of the present disclosure. The table 600 shows different indoor metrics 605 such as carbon dioxide, ozone, total VOCs, PM₁₀, PM_{2.5}, PM₁, carbon monoxide, nitrogen dioxide, sulfur dioxide, relative humidity, and temperature. A health impact 610 on the immune system, respiratory disease, cell/tissue damage, organ dysfunction, and microbiome disruption may be shown as a function of each indoor metric and classified (by hashing) as low health hazard and impact 620, moderate health hazard and impact 630, and high health hazard and impact 640. Thus, medical and/or toxicity data may be used to understand how the indoor air metrics affect human health.

In some embodiments, once different pollutant concentrations are determined from the plurality of sensors and the health impact scores may be computed, at least one verified environmental hazard type related to any of the pollutants in FIG. 8, for example, may be determined at the site location when at least one particular health impact score of the plurality of health impact scores is less than a respective predefined threshold score that is unique to the at least one particular environmental parameter (e.g., concentrations and/or levels of particulate matter PARTICLES, carbon monoxide CO, carbon dioxide CO₂, nitrous dioxide NO₂, sulfur dioxide SO₂, total volatile organic compound tVOCs, temperature TEMP, ozone, humidity).

In some embodiments, the processor 20 and/or the sensor based computing device processor 75 may use the computed set of health impact scores {HI(PM₁₀), HI(PM_{2.5}), HI(PM₁), HI(tVOC), HI(NO₂), HI(SO₂), HI(CO), HI(O3), HI(CO₂), HI(RH), HI(Temp)} for each of the different pollutant types to determine if at least one environmental hazard type is present in the site location. The processor 20 and/or the SB computing device processor 75 may determine if there is at least one verified environmental hazard type when at least one particular health impact score in the set of HI scores is less than a predefined threshold score that is unique to the at least one particular environmental parameter or pollutant. For example, the predefined threshold score may be different or unique for each pollutant in the set of HI scores. In other embodiments, the predefined threshold score may be the same threshold score for all of the different pollutants.

For example, a health hazard may exist for carbon dioxide when the HI score for carbon dioxide is below a predefined score of 50, whereas the health hazard may exist for ozone when the HI score for ozone is below a predefined score of 70. When HI for a particular pollutant is below the predefined threshold score, then that pollutant become a verified environmental hazard type, where there are carbon dioxide concentrations or sulfur dioxide concentrations, for example, that are too high in the site location and more specifically in the indoor environment at the site location.

In some embodiments, the machine learning model 28 may generate at least one recommendation for remediating the at least one verified environmental hazard type when inputting the plurality of health impact scores {HI(PM₁₀), HI(PM_{2.5}), HI(PM₁), HI(tVOC), HI(NO₂), HI(SO₂), HI(CO), HI(O3), HI(CO₂), HI(RH), HI(Temp)} into the at least one machine learning model. The at least one recommendation may be executed manually by the user 90, for example, or as shown in the embodiments of FIG. 2, the at least one remediation recommendation may be used to generate instructions which may be transmitted by the ECE manager 35 over the communication network 30 to automatically control at least one environment-controlling equipment located at the at least one site location so as to change an operational parameter of the at least one environment-controlling equipment to mitigate the at least one verified environmental hazard type.

In some embodiments, the machine learning model 28 may be, for example, a classification neural network model trained to map the set of health impact scores to remediation recommendations and/or remediation actions for mitigating the at least one verified environmental hazard type as described below with reference to Table I.

In some embodiments, the machine learning model 28 may be, for example, a classification neural network model trained to map the set of health impact scores and/or personalized medical data for occupants in the site location to personalized remediation recommendations and/or remediation actions for mitigating the at least one verified environmental hazard type as described below with reference to Table I.

In some embodiments, a computer-controlled window actuator (e.g., an environment-controlling equipment) may have the window set to be closed, where the closed window is an operational parameter of the environment-controlling equipment. The SB computing device may detect, for example, that the concentration of tVOCs is too high, the processor 20 and/or the sensor-based computing device processor 75 may send instructions via the ECE manager 35 and/or ECE controller 85 to change the operational parameter of the environment-controlling equipment (e.g., initially closed window and/or closed vent 120) by opening the window 150 and/or opening the ceiling vent 120 and/or activating the ceiling fan 135, for example.

In some embodiments, the following Table I is an exemplary output from the machine learning model 28 that may be used to generate instructions to transmit to the at least one environment-controlling equipment located at the site location. The computer-controlled equipment may include actuated environmental control devices, for example, with reference to FIG. 2. The instructions may trigger the computer-controlled actuators in the equipment to automatically increase or decrease outdoor air fraction in the room by controlling the vent 120, disperse indoor air in the room by controlling the fan 135, raise or lower a temperature of the room by controlling the thermostat 110, raise or lower humidity in the room by controlling the humidifier/dehumidifier 130, activate or deactivate air cleaners in the room by controlled the air filter 160, or any combination thereof.

In some embodiments, the machine learning model 28 may be used to determine which action recommendations are most effective in raising the health impact scores, to predict the times of day that certain pollutant variables are likely to have certain concentrations so that the users may take preventive actions, and/or to fine tune the sensor data according to evolving research.

The remediation recommendation actions generated by the machine learning model 28 for the different pollutants are shown below in Table I:

TABLE I
Remediation recommendation actions (in bold)

Particles
Dilute
If (indoor RH < 40% && outdoor RH > indoor RH && outdoor HIR >=
indoor HIR):
“Increase outside air fraction to dilute indoor particles”
If (indoor RH < 40% && outdoor RH < indoor RH):
“Decrease outdoor air fraction and turn on humidifier (if
available) to decrease indoor particles”
If (indoor RH > 60% && outdoor RH < indoor RH && outdoor HIR >=
indoor HIR):
“Increase outdoor air fraction to dilute indoor particles”
If (outdoor HIR > indoor HIR):
“Increase outdoor air fraction to dilute indoor particles”
Disperse
If (outdoor HIR < indoor HIR):
“Decrease outdoor air fraction and increase exhaust rate (if
available) to disperse indoor particles”
Remove
If (other remediations don't work):
“Replace/Upgrade filters on existing air handling units (if
available) to decrease indoor particles”
tVOCs
Dilute
If (indoor RH < 40% && outdoor RH > indoor RH && outdoor HIR >=
indoor HIR):
“Increase outdoor air fraction to dilute total Volatile Organic
Compounds”
If (indoor RH > 60% && outdoor RH < indoor RH && outdoor HIR >=
indoor HIR):
“Increase outdoor air fraction to dilute total Volatile Organic
Compounds”
If (indoor RH > 60% && outdoor RH > indoor RH):
“Decrease outdoor air fraction and dehumidify to return
Relative Humidity to between 40-60%”
If (indoor temp < 72 degrees F):
“Increase indoor temperature up to 72° F. to return Relative
Humidity to between 40-60%”
If (outdoor HIR > indoor HIR):
“Increase outdoor air fraction to dilute total Volatile Organic
Compounds”
Disperse
If (outdoor HIR < indoor HIR):
“Decrease outdoor air fraction and increase exhaust (if
available) to disperse total Volatile Organic Compounds”
RH
If (indoor RH > 60% && outdoor RH < 40% && outdoor HIR >= indoor HIR):
“Increase outdoor air fraction to increase ventilation to return Relative
Humidity to between 40-60%”
If (indoor RH > 60% && outdoor RH > 60%):
“Turn on dehumidifier (if available) or increase the temperature to
return Relative Humidity to between 40-60%”
If (indoor RH < 40% && outdoor RH > 40%):
“Increase outdoor air fraction to return Relative Humidity to between
40-60%”
If (indoor RH < 40%)
“Turn on humidifier (if available) or lower the temperature to return
Relative Humidity to between 40-60%”
Temperature
Too high
If (outdoor temperature is between 68-73° F. && outdoor HIR >= indoor
HIR):
“Increase outdoor air fraction to decrease temperature.
Maintain a temperature between 68-73° F.”
Or else:
“Decrease the temperature. Maintain a temperature between 68-
73° F.”
Too low
If (outdoor temperature is between 68-73° F. && outdoor HIR >= indoor
HIR):
“Increase outdoor air fraction to increase temperature.
Maintain a temperature between 68-73° F.”
Or else:
“Increase the temperature. Maintain a temperature between 68-
73° F.”
CO₂
Dilute
If (outdoor HIR > indoor HIR):
“Increase outdoor air fraction to dilute Carbon Dioxide”
Disperse
If (outdoor HIR < indoor HIR):
“Decrease outdoor air fraction and increase exhaust (if
available) to disperse Carbon Dioxide”
O₃
Dilute
If (outdoor HIR > indoor HIR):
“Increase outdoor air fraction to dilute Ozone”
Disperse
If (outdoor HIR < indoor HIR):
“Decrease outdoor air fraction and increase exhaust (if
available) to disperse Ozone”
Control Source
If (Dilute and Disperse don't work):
“Turn off electronic devices that may be producing Ozone”
CO
Dilute
If (outdoor HIR > indoor HIR):
“Increase outdoor air fraction to dilute Carbon Monoxide”
Disperse
If (outdoor HIR < indoor HIR):
“Decrease outdoor air fraction and increase exhaust (if
available) to limit Carbon Monoxide infiltration”
Control Source
If (Dilute and Disperse don't work):
“Find and eliminate any source of indoor gas production (for
example cooking fumes or fire in fireplace)”
NO₂
“Decrease outdoor air fraction and increase exhaust (if available)
to limit Nitrogen Dioxide infiltration”
SO₂
“Decrease outdoor air fraction and increase exhaust (if available)
to limit Sulfur Dioxide infiltration”

In some embodiments, the remediation recommendation actions shown in bold in Table I may cause the processor 20 to transmit first instructions to the SB computing device 70 to display on the display 80 and/or on the GUI 96, a remediation action message and/or to sound an alarm, for example. In other embodiments, the remediation recommendation actions shown in bold in Table I may cause the processor 20 using the ECE manager 35 to transmit second instructions to the ECE controller 85 to automatically change the operational parameter of the environment-controlling equipment as previously described.

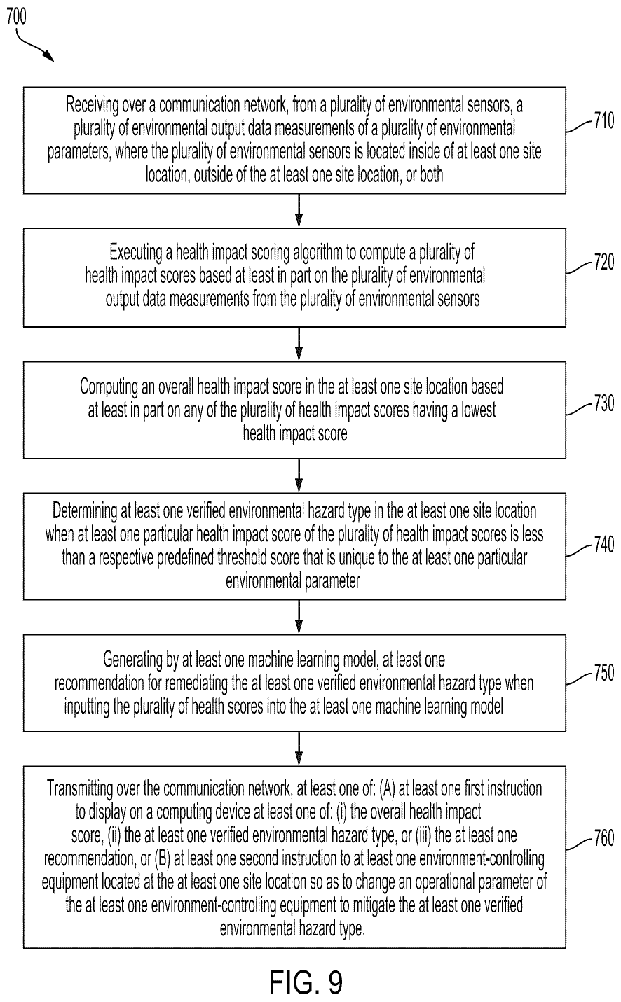

FIG. 9 illustrates a flowchart of an exemplary method 700 for assessing and managing a health impact of an indoor environment at a site location in accordance with one or more embodiments of the present disclosure. The method may be performed, for example, by the processor 20.

The method 700 may include receiving 710 over a communication network, from a plurality of environmental sensors, a plurality of environmental output data measurements of a plurality of environmental parameters, where the plurality of environmental sensors is located inside of at least one site location, outside of the at least one site location, or both.

The method 700 may include executing 720 a health impact scoring algorithm to compute a plurality of health impact scores based at least in part on the plurality of environmental output data measurements from the plurality of environmental sensors. A first exemplary embodiment of the health impact scoring algorithm 24 may be given by Equations (1)-(3) where each of the health impact scores for each of the different pollutants may be further based on any of an ozone factor, a relative humidity factor, and/or predefined pollutant-specific breakpoint hazard values as shown for example in FIG. 6. A second exemplary embodiment of the health impact scoring algorithm 24 may be further based on using a value from each of the plurality of environmental output data measurements and a square of the value.

The method 700 may include computing 730 an overall health impact score in the at least one site location based at least in part on any of the plurality of health impact scores having a lowest health impact score.

The method 700 may include determining 740 at least one verified environmental hazard type in the at least one site location when at least one particular health impact score of the plurality of health impact scores is less than a respective predefined threshold score that is unique to the at least one particular environmental parameter.

The method 700 may include generating 750 by at least one machine learning model, at least one recommendation for remediating the at least one verified environmental hazard type when inputting the plurality of health impact scores into the at least one machine recommendation learning model.

The method 700 may include transmitting 760 over the communication network, at least one of: (A) at least one first instruction to display on a computing device at least one of: (i) the overall health impact score, (ii) the at least one verified environmental hazard type, or (iii) the at least one recommendation, or (B) at least one second instruction to at least one environment-controlling equipment located at the at least one site location so as to change an operational parameter of the at least one environment-controlling equipment to mitigate the at least one verified environmental hazard type.

The sensor-based computing device 70 as disclosed herein provides a technical solution for harmonizing a common sensing process using multiple environmental sensors to automatically assess a health impact of an indoor environment at least one site location by computing a plurality of health impact scores based on the multiple environmental sensor output data. If the system 10 using the sensor-based computing device 70 verifies that at least one verified environmental hazard type exists, the system 10 may automatically implement measures for remediating the at least one verified environmental hazard type by integrating the use of the computed health impact scores to automatically cause changes in operational parameter(s) of environment-controlling equipment to mitigate the at least one verified environmental hazard type at least one site location.

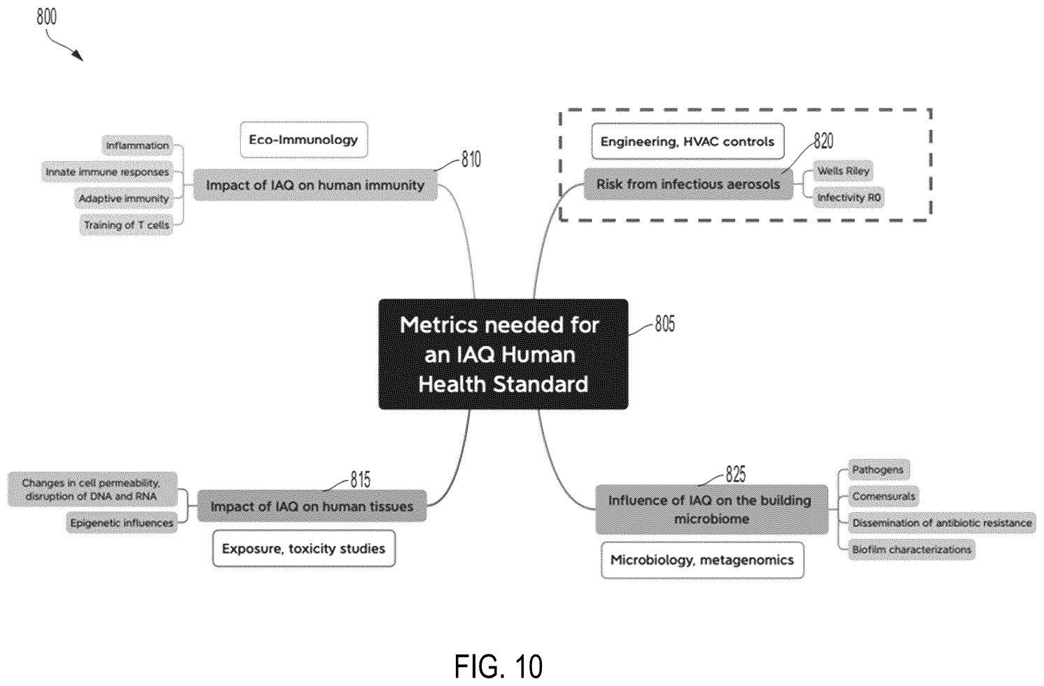

FIG. 10 is a diagram 800 showing various metrics used for formulating an indoor air quality (IAQ) human health standard 805 in accordance with one or more embodiments of the present disclosure. IAQ standards for human health have moved beyond typical heating, ventilation, and/or air conditioning (HVAC) controls that consider comfort levels and/or risks from infectious aerosols 820 to now consider three additional components of the indoor environment on human health: (1) an impact of IAQ on human immunity 810, (2) an impact of IAQ on human tissues 815 (e.g., how individual gases and/or particles and combinations with each other diffuse through cells and affect human physiology), and (3) an impact of IAQ on the building microbiome 825 (e.g., how indoor environment in the building affects the microbial populations).

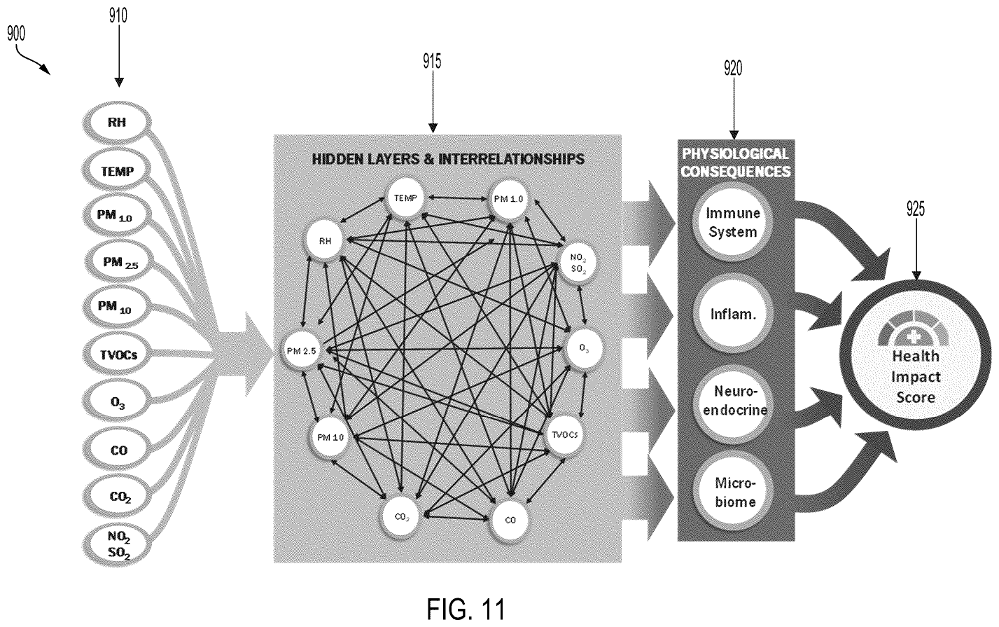

FIG. 11 is a second flow diagram 900 showing an algorithm 915 for computing an overall health impact score 925 in accordance with one or more embodiments of the present disclosure. The algorithm 915 may be executed by the processor 20. The plurality of environmental sensors may generate a plurality of environmental output data measurements 910 (e.g., 11 different pollutant metrics shown in FIG. 11) that are input to the health impact scoring algorithm 915. In some embodiments, the health impact scoring algorithm 915 may include a machine learning model trained to account for the interrelationships between all of the different pollutant metrics on each individual pollutant health impact score. In other embodiments, the health impact scoring algorithm 915 may include one or more algorithms that account for the interrelationships between all of the different pollutant metrics on each individual pollutant health impact score. In yet other embodiments, the health impact scoring algorithm 915 may include at least one algorithm and at least one trained machine learning model. The output of the health impact scoring algorithm 915 account for these interrelationships may be account for physiological consequences 920 (e.g., immune system, inflammation, neuro-endocrine, and/or microbiome) in computing the health impact score 925.

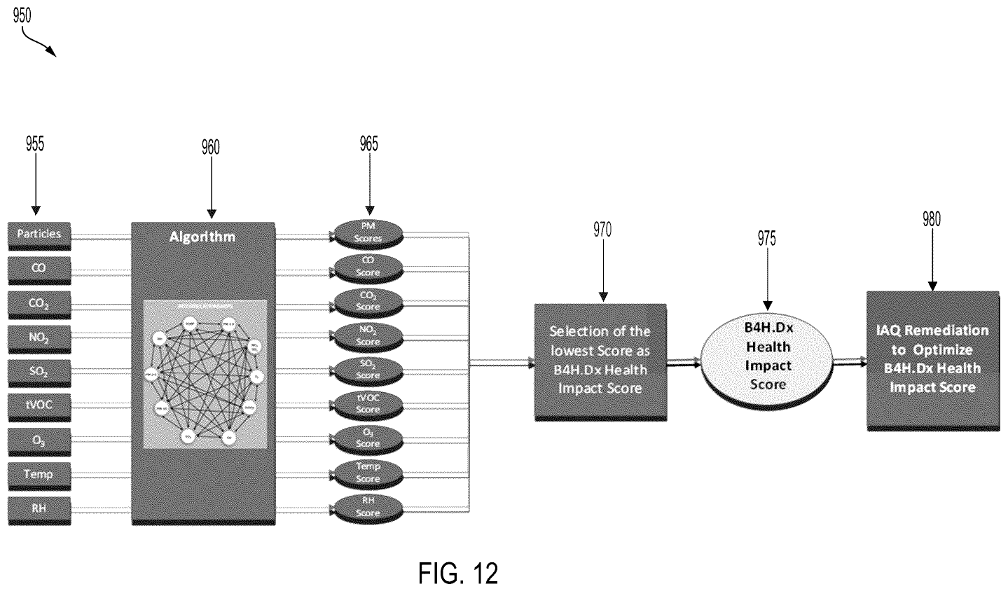

FIG. 12 is a third flow diagram 950 showing an algorithm 960 for computing a set of individual health impact scores 965 in accordance with one or more embodiments of the present disclosure. The algorithm 960 may be executed by the processor 20. The plurality of environmental sensors may generate a plurality of environmental output data measurements 955 that are input to the health impact scoring algorithm 960.

In some embodiments, the health impact scoring algorithm 960 may include a machine learning model trained to account for the interrelationships between all of the different pollutant metrics on each of the individual pollutant health impact scores 965. In other embodiments, the health impact scoring algorithm 960 may include one or more algorithms that account for the interrelationships between all of the different pollutant metrics on each individual pollutant health impact score 965. In yet other embodiments, the health impact scoring algorithm 960 may include at least one algorithm and at least one trained machine learning model.

In some embodiments, the interactions between pollutants, or pollutants and thermal conditions, that result in increased or decreased health impacts may be accounted for in the algorithm(s). The increased or decreased health impact may be a consequence of interactions that alter pollutant penetration into human tissues or change the degree of reactivity with cellular and sub-cellular components. Examples include but are not limited to interactions that may lead to changes in water solubility, size limited tissue penetration, cell membrane diffusability, oxidation potential, DNA intercalation and/or epigenetic disruption.

In some embodiments, the processor 20 may select 970 the lowest score of the individual pollutant health impact scores 965 to determine an overall health impact score 975.

In some embodiments, the processor 20 may use the overall health impact score 975 based on the lowest pollutant health impact score 965 for a particular pollutant to execute a machine learning model (e.g., MLM 28) trained to output remediation recommendations (e.g., remediations recommendations 30 from FIG. 1) to optimize the health impact score 975. Note that if the overall health impact score 975 is low due to a plurality of pollutants in a particular indoor environment, the processor 20 may iteratively optimize the indoor environment to reduce the effect of each pollutant from the plurality of pollutants until the overall the health impact score 975 is within an acceptable range.

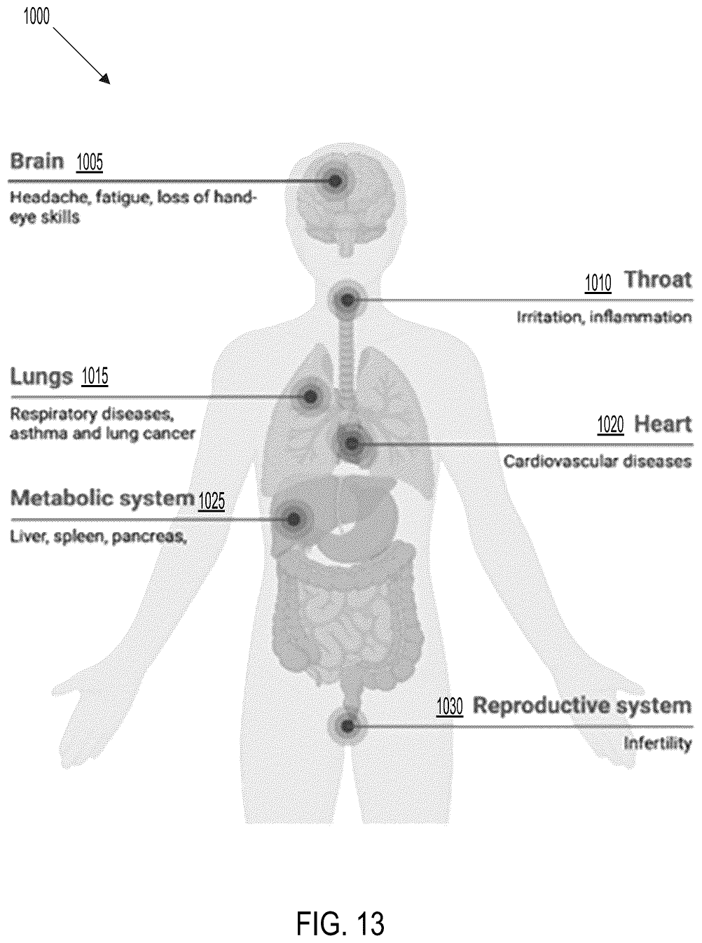

FIG. 13 is a diagram 1000 illustrating quantifiable health effects of primary and interacting indoor pollutants as well as thermal conditions in accordance with one or more embodiments of the present disclosure. For example, primary and interacting indoor pollutant exposure: (1) in a brain 1005 may cause headache, fatigue, and/or loss of hand-eye skills, (2) in a throat 1010, may cause irritation and inflammation, (3) in lungs 1015, may cause respiratory diseases, asthma, and lung cancer, (4) in a heart 1020, may cause cardiovascular diseases, (5) in a metabolic system 1025, may cause problems with liver, spleen, and/or pancreas, and/or (6) in a reproductive system 1030, may cause infertility.

In some embodiments, with regard to process modelling for indoor air and health impact rating, pollutants in the environment may enter the body in three ways: inhalation, skin or mucus membrane contact and subsequent penetration to underlying tissues, and ingestion. The health score may report the impact of inhaled pollutants within the thermal conditions present on human occupant physiological systems. The B4H scoring may be based on an extensive database from epidemiology, toxicology, cell biology, microbiome research and indoor chemistry.

In some embodiments, with regard to indoor gases and occupant health, when a building occupant inhales a gaseous pollutant, the degree and site of damage to the occupant's physiology may depend on the gas concentration, water solubility (degree of polarity) of the gas phase molecule, and the ability of the molecule to react and oxidize tissues. When inhaled, a gas that is highly water soluble (polar) may quickly dissolve into the upper respiratory tract mucus layer where it may cause local irritation and/or damage to the nearby cells lining the sinuses, nose, throat, larynx, and/or upper bronchial tubes in the lungs. Conversely, inhaled gases that are insoluble in water (non-polar) may be carried by mucus into deeper lung regions while maintaining their original molecular configuration. For example, sulfur dioxide (SO2), highly soluble in water, may damage the upper airways and skin, whereas less water-soluble nitrogen dioxide (NO2) and ozone (O3) may penetrate and cause damage in deeper lung regions. The characteristics of the initial gas, and well as molecular changes from reactions with other indoor pollutants that may change the polarity and reactivity, may influence the degree of harm to occupant health.

In some embodiments, with regard to airborne particles and occupant health, particles may be present in the air and may be inhaled. Harm from particles may depend on their size and reactivity with cell membranes. For example. larger particles with diameters greater than 10 microns (PM₁₀) may be trapped by mucus membranes of the upper airways where they may trigger coughing. Fine particles (PM_{2.5}) may be swept into deep regions of the respiratory system and may trigger bronchial spasm manifested by wheezing and coughing. Ultrafine particles (PM0.1) may pass through the lung—capillary membrane and may be carried via the circulatory system to all cells in the body. Therefore, smaller particles may penetrate more deeply and may have greater systemic toxicity.

In some embodiments, when particles encounter cell walls, inflammation may occur followed by a cascade of effects. For example, particles in lung tissue may cause inflammation, followed by oxidative stress. Oxidative stress may activate pro-inflammatory signaling and depletion of antioxidants, setting off a cascade of events that may affect distant organs. Repeated exposure to particle pollution and consequent inflammation may contribute to cardiovascular conditions such as atherosclerosis as well as metabolic disorders. Ultrafine particles that go directly into different organs may trigger inflammation in that organ.

In some embodiments, molecular structure may determine exposure pathways: (A) Water soluble, non-diffusible polar compounds and solid particles >1.0 microns diameter and penetration factors, and (B) non-water soluble, diffusible lipid soluble and solid particles with <1.0 micron diameters and permeation reactions. Note that both (A) and (B) categories may be influenced by enthalpy exchange measured as sensible and latent heat loads. Thus, indoor exposures may have a quantifiable effect on occupant physiology determined by both the primary pollutant as well as by cellular consequences of combined pollutants.

In some embodiments, the health effects of indoor pollutants and thermal conditions on at least one occupant in a given site location may be quantified, for example, using the health impact score for a particular pollutant (e.g., FIG. 7), which is based on the pollutant concentration for the particular pollutant measured by the sensors in the given site location, and an exposure time of the particular pollutant at the pollutant concentration in the given site location.

In some embodiments, the processor 20 via the communication network 30 may be configured to determine the identity of each of a plurality of occupants in a site location and to track a personal exposure time for each occupant to at least one particular pollutant at the site location. For example, the identity of each of the plurality of occupants and the time that each of the plurality of occupants is present in the site location may be determined by facial recognition techniques (e.g., cameras) deployed in the site location, using identification cards to enter the site location such as via an ID card reader at the entrance to the site location, personal wearable devices with location tracking circuitry and/or personal cellphone devices with location tracking circuitry or any suitable method for identifying each of the plurality of occupants. The processor 20 may then track the exposure time of each occupant to any of the plurality of pollutants as shown in FIGS. 5-8.

In some embodiments, the processor 20 may be configured to track a cumulative personalized exposure time of a particular person to at least one particular pollutant at a plurality of site locations (e.g., as the particular person moves between different site locations such as different rooms in a building and/or different buildings in a particular geographic region) with sensor-based computing devices.

In some embodiments, the processor 20 may be configured transmit instructions to display on the N GUIs 80A . . . 80B, possible health effects on the at least one occupant in the site location that the particular verified environmental hazard type was measured based on the exposure time of the at least one occupant to each particular verified environmental hazard type. For example, if a concentration level of particulate matter in the indoor air at the site location 21A causes the HIR to drop to a hazardous level, for example, the GUI 1 80A may display “THE PARTICULATE MATTER LEVEL IN THE AIR IS AT HAZARDOUS LEVELS AND MAY INDUCE RESPIRATORY DISEASES FOR PROLONGED EXPOSURE”

In some embodiments, the processor 20 may be configured to generate a personalized report for each occupant at the site location, where the personalized report may include a personal exposure time that each occupant was exposed to each particular verified environmental hazard type, health effects on each occupant based on the personal exposure time that each occupant was exposed to each particular verified environmental hazard type, and/or recommendations for each occupant to obtain medical help. For example, the processor 30 may generate a generalized report for a specific occupant such as for example, “OUR SYSTEM DETERMINED THAT YOU WERE EXPOSED TO A HAZARDOUS LEVEL OF PARTICULATES FOR A TOTAL OF <x> HOURS FROM <START DATE> TO <END DATE>. NOTE THAT THE EXPOSURE TIME OF <X> HOURS MAY CAUSE RESPIRATORY PROBLEMS AND IT IS RECOMMENDED THAT YOU HAVE PULMONARY AND/OR BLOOD TESTS PERFORMED TO DETERMINE IF THERE WAS ANY RESPIRATORY DAMAGE”.

In some embodiments, the processor 20 of the server 15 and/or the processor 91 of the mobile computing device 91 may use a trained machine learning model 28 and/or a trained machine learning model 93 that is configured to output personalized health recommendations 31 of a particular occupant when the personal medical data (P.INFO) 94 of the particular occupant is available on the mobile computing device 95 and/or in the personal database 66 in the server 15. For example, if the personal medical data 94 indicates the particular occupant suffers from severe asthma and is required to carry a steroidal inhaler in the event of an allergy attack. The machine learning model may be trained to determine how long a person with a severe asthmatic condition may withstand breathing the air with a high level of particulates.

In some embodiments, standalone physiological sensors and/or a smartphone including physiological sensors and/or a wearable device including physiological sensors may be used to measure real-time physiological data of an occupant. The real-time physiological data of the occupant may be used as additional inputs to the machine learning models for determining the personalized health recommendations 31 of a particular occupant. The physiological sensors may include, for example, but are not limited to physiological sensors such as pulse, heart rate (HR), electrocardiogram (ECG), electroencephalogram (EEG), blood pressure (BP), electromyography (EMG), Pulse oximetry, photoplethysmogram (PPG), accelerometer (ACC), Glucose, Gyroscope, motion tracker and/or body temperature.

In some embodiments, the processor 20 executing the MLM 28 and/or the processor 91 executing the MLM 93 may be further configured to output a personalized recommendation when the HI value for any of the particulates drops to a hazardous level. The personalized recommendation may be outputted on the GUI 96 as “JOHN, THE ROOM YOU ARE IN HAS A VERY HAZARDOUS POLLUTANT LEVEL WHICH MAY TRIGGER A SEVERE ASTHMA ATTACK AFTER <a first predetermined allowable exposure time> MINUTES. PLEASE LEAVE THE ROOM IMMEDIATELY AND/OR WEAR AN N95 MASK AND/OR USE YOUR INHALER TO SELF ADMINISTER YOUR ANTI-ASTHMA MEDICATION NOW.”

In some embodiments, the predetermined allowable exposure time to a particular health hazard such as for example, a hazardous particulate level in the air of the indoor environment for a healthy occupant may be one hour as compared to 5 minutes (e.g., the first predetermined allowable exposure time) for the asthmatic occupant in the exemplary embodiment described above. The machine learning models and/or algorithms may be trained to output the predetermined allowable exposure time for use in personalized recommendations when the health impact scores measured by the SB computing device are inputted into the model with the personal medical data of the plurality of occupants.

In some embodiments, the recommendations from the outputted machine learning models may be transmitted to the environmental controlling equipment (ECE) manager 35 that is configured to send instructions to at least one environmental controlling equipment for mitigating the at least one verified environmental hazard type so as to cause the corresponding health impact score to improve. As the health impact score associated with the at least one verified environmental hazard type improves, the personalized recommendations may change dynamically. For example, in the case of particulate matter, if the health impact score is hazardous and the ECE manager 35 triggers an activation of the ceiling fan 135 to disperse the indoor air that changes the health impact rating from hazardous to acceptable in a matter of two minutes, for example, the personalized recommendation may be outputted on the GUI 96 may change from the hazardous case shown above to “JOHN, THE ROOM YOU ARE IN HAS AN ACCEPTABLE POLLUTANT LEVEL BUT MAY STILL TRIGGER AN ASTHMA ATTACK AFTER <a second predetermined allowable exposure time> MINUTES. YOU MAY CONSIDER LEAVING THE ROOM AND/OR WEAR A MASK AND/OR USE YOUR INHALER TO SELF ADMINISTER YOUR ANTI-ASTHMA MEDICATION NOW.” After 2 minutes after activating the ceiling fan 135, the second predetermined allowable exposure time may now be determined by the machine learning models and/or algorithms for the asthmatic to be 20 minutes for the acceptable pollutant level instead of 5 minutes for the hazardous pollutant level.

In some embodiments, processor 20 may be configured to automatically transmit the personalized reports for each occupant from the plurality of occupants over the communication network 30 to a computing device associated with each occupant (e.g., the mobile computing device 95 associated with the user 90), to a computing device associated with an employer, to a computing device associated with an insurance company, and/or to a computing device associated with a medical provider.

In some embodiments, the processor 20 executing the MLM 28 and/or the processor 91 executing the MLM 93 may be further configured to determine using location tracking data and the at least one exposure time that the at least one verified environmental hazard type was present in the site location, at least one personal exposure time that each occupant has been exposed to the at least one verified environmental hazard type in the site location.

In some embodiments, the processor 20 executing the MLM 28 and/or the processor 91 executing the MLM 93 may be further configured to generate a personalized report for each occupant at the site location, where the personalized report may include the at least one personal exposure time that each occupant was exposed to the at least one verified environmental hazard type, and at least one possible health effect on each occupant based on the at least one personal exposure time that each occupant has been exposed to the at least one verified environmental hazard type.

In some embodiments, the processor 20 executing the MLM 28 and/or the processor 91 executing the MLM 93 may be further configured to transmit an instruction to a computing device to display a personalized alert associated with each occupant at the site location, where the personalized alert may include a notification of a presence of the at least one verified environmental hazard type in the site location, and the at least one personalized recommendation for reducing an exposure to the at least one verified environmental hazard type in the site location.

The exemplary embodiments for the generation of personalized reports/recommendations for a plurality of occupants in an indoor environment of a site location having hazardous particulate levels is shown hereinabove merely for conceptual clarity and not by way of limitation of the embodiments disclosed herein. Any of the pollutants discussed hereinabove such as in FIGS. 5-8 may be used and the personalized reports/recommendations may be adapted accordingly. For example, if the pollutant is VOCs, the personalized reports/recommendations may indicate that a prolonged exposure to VOCs may cause brain damage, or worse, death.

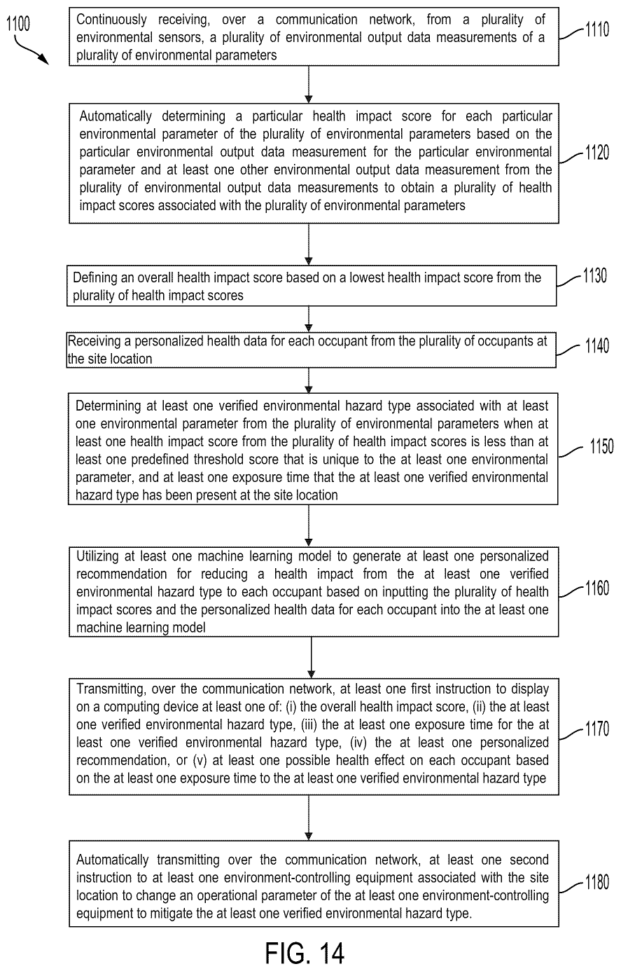

FIG. 14 illustrates a flowchart of an exemplary method 1100 for assessing and mitigating personal health hazards in an indoor environment at a site location for a plurality of occupants in accordance with one or more embodiments of the present disclosure. The method may be performed, for example, by the processor 20 and/or the processor 91.

The method 1100 may include continuously receiving 1110, over a communication network, from a plurality of environmental sensors, a plurality of environmental output data measurements of a plurality of environmental parameters.

The method 1100 may include automatically determining 1120 a particular health impact score for each particular environmental parameter of the plurality of environmental parameters based on the particular environmental output data measurement for the particular environmental parameter and at least one other environmental output data measurement from the plurality of environmental output data measurements to obtain a plurality of health impact scores associated with the plurality of environmental parameters.

The method 1100 may include defining 1130 an overall health impact score based on a lowest health impact score from the plurality of health impact scores.

The method 1100 may include receiving 1140 a personalized health data for each occupant from the plurality of occupants at the site location.

The method 1100 may include determining 1150 at least one verified environmental hazard type associated with at least one environmental parameter from the plurality of environmental parameters when at least one health impact score from the plurality of health impact scores is less than at least one predefined threshold score that is unique to the at least one environmental parameter, and at least one exposure time that the at least one verified environmental hazard type has been present at the site location.

The method 1100 may include utilizing 1160 at least one machine learning model to generate at least one personalized recommendation for reducing a health impact from the at least one verified environmental hazard type to each occupant based on inputting the plurality of health impact scores and the personalized health data for each occupant into the at least one machine learning model.

The method 1100 may include transmitting 1170, over the communication network, at least one first instruction to display on a computing device at least one of: (i) the overall health impact score, (ii) the at least one verified environmental hazard type, (iii) the at least one exposure time for the at least one verified environmental hazard type, (iv) the at least one personalized recommendation, or (v) at least one possible health effect on each occupant based on the at least one exposure time to the at least one verified environmental hazard type.

The method 1100 may include automatically transmitting 1180, by the processor, over the communication network, at least one second instruction to at least one environment-controlling equipment associated with the site location to change an operational parameter of the at least one environment-controlling equipment to mitigate the at least one verified environmental hazard type.

In some embodiments, exemplary inventive, specially programmed computing systems/platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiments, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a social media post, a map, an entire application (e.g., a calculator), etc. In some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21) .NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, exemplary inventive computer-based systems/platforms, exemplary inventive computer-based devices, and/or exemplary inventive computer-based components of the present disclosure may be configured to handle numerous concurrent sensor-based computing devices that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, and/or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, the terms “proximity detection,” “locating,” “location data,” “location information,” and “location tracking” refer to any form of location tracking technology or locating method that can be used to provide a location of, for example, a particular computing device/system/platform such as the location tracking (LT) circuitry 97 of the present disclosure and/or any associated computing devices, based at least in part on one or more of the following techniques/devices, without limitation: accelerometer(s), gyroscope(s), Global Positioning Systems (GPS); GPS accessed using Bluetooth™; GPS accessed using any reasonable form of wireless and/or non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

In some embodiments, the communication circuitry 50, 82A, and 82B, for example, may be equipped to communicate over a wired and/or wireless communication medium (e.g., NFC, RFID, NBIOT, 3G, 4G, 5G, GSM, GPRS, WiFi, WiMax, CDMA, satellite, ZigBee, etc.).

In some embodiments, the communication circuitry 50, 82A, and 82B, for example, may provide network access, data transport and/or other services to any computing device coupled to it. In some embodiments, the communication network 30 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the communication network 30 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the communication network may include and implement, as an alternative or in conjunction with one or more of the above, a WiMAX architecture defined by the WiMAX forum. In some embodiments and, optionally, in combination of any embodiment described above or below, the communication network 30 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the communication network 30 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the communication network 30 may also include mass storage, such as network attached storage (NAS), a storage area network (SAN), a content delivery network (CDN) or other forms of computer or machine readable media.

In some embodiments, the server 15 may be a web server (or a series of servers) running a network operating system, examples of which may include but are not limited to Microsoft Windows Server, Novell NetWare, or Linux. In some embodiments, the server 15 may be used for and/or provide cloud and/or network computing.

In some embodiments, the server 15 includes a computer-readable medium, such as a random-access memory (RAM) (e.g., the memory 55) coupled to a processor or FLASH memory (e.g., the processor 20). In some embodiments, the processor 20 may execute computer-executable program instructions stored in the memory. In some embodiments, the processor 20 may include a microprocessor, an ASIC, and/or a state machine. In some embodiments, the processor may include, or may be in communication with, media, for example computer-readable media, which stores instructions that, when executed by the processor 20, may cause the processor 20 to perform one or more steps described herein. In some embodiments, examples of computer-readable media may include, but are not limited to, an electronic, optical, magnetic, or other storage or transmission device capable of providing a processor, such as the processor 20, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, the I/O devices 45 and/or the I/O devices 77A and 77B may also comprise a number of external or internal devices such as a mouse, a CD-ROM, DVD, a physical or virtual keyboard, a display, a speaker, or other input or output devices.

In some embodiments, at least one database of exemplary databases 60 and 65 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

In some embodiments, the exemplary inventive computer-based systems/platforms, the exemplary inventive computer-based devices, and/or the exemplary inventive computer-based components of the present disclosure may be configured to utilize one or more exemplary AI/machine learning techniques (e.g., the machine learning model 28) chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

At least some aspects of the present disclosure will now be described with reference to the following numbered clauses.

1. A method may include:
receiving, by a processor, over a communication network, from a plurality of environmental sensors, a plurality of environmental output data measurements of a plurality of environmental parameters;
   where the plurality of environmental sensors may be located inside of at least one site location, outside of the at least one site location, or both;
   wherein the plurality of environmental sensors may include:
   (i) an ozone sensor, outputting an ozone output data measurement of the plurality of environmental output data measurements,
   (ii) a humidity sensor, outputting a humidity output data measurement of the plurality of environmental output data measurements,
   (iii) a temperature sensor, outputting a temperature output data measurement of the plurality of environmental output data measurements,
   (iv) a carbon dioxide sensor, outputting a carbon dioxide output data measurement of the plurality of environmental output data measurements,
   (v) a carbon monoxide sensor, outputting a carbon monoxide output data measurement of the plurality of environmental output data measurements,
   (vi) a nitrous dioxide sensor, outputting a nitrous dioxide output data measurement of the plurality of environmental output data measurements,
   (vii) a sulfur dioxide sensor, outputting a sulfur dioxide output data measurement of the plurality of environmental output data measurements,
   (viii) a total volatile organic compound (tVOC) sensor, outputting a tVOC output data measurement of the plurality of environmental output data measurements, and
   (ix) at least one particulate matter sensor, outputting at least one particulate matter output data measurement of the plurality of environmental output data measurements;
executing, by the processor, a health impact scoring algorithm to compute a plurality of health impact scores based at least in part on the plurality of environmental output data measurements from the plurality of environmental sensors;
   where a humidity health impact score of the plurality of health impact scores may be based at least in part on the humidity output data measurement;
   where an ozone health impact score of the plurality of health impact scores may be based at least in part on the ozone output data measurement;
   where a temperature health impact score of the plurality of health impact scores may be based at least in part on the temperature output data measurement;
   where a carbon dioxide health impact score of the plurality of health impact scores may be based at least in part on the carbon dioxide output data measurement;
   where a carbon monoxide health impact score of the plurality of health impact scores may be based at least in part on the carbon monoxide output data measurement;
   where a nitrous dioxide health impact score of the plurality of health impact scores may be based at least in part on the nitrous dioxide output data measurement;
where a sulfur dioxide health impact score of the plurality of health impact scores may be based at least in part on the sulfur dioxide output data measurement;
where a tVOC health impact score of the plurality of health impact scores may be based at least in part on the tVOC output data measurement;
   where at least one particulate matter health impact score of the plurality of health impact scores may be based at least in part on the at least one particulate matter output data measurement;
computing, by the processor, an overall health impact score in the at least one site location based at least in part on any of the plurality of health impact scores having a lowest health impact score;
determining, by the processor, at least one verified environmental hazard type in the at least one site location when at least one particular health impact score of the plurality of health impact scores is less than a respective predefined threshold score that is unique to the at least one particular environmental parameter;
generating, by the processor, by at least one machine learning model, at least one recommendation for remediating the at least one verified environmental hazard type when inputting the plurality of health impact scores into the at least one machine recommendation learning model;
transmitting, by the processor, over the communication network, at least one of:
   (A) at least one first instruction to display on a computing device at least one of:
      (i) the overall health impact score,
      (ii) the at least one verified environmental hazard type, or
      (iii) the at least one recommendation, or
   (B) at least one second instruction to at least one environment-controlling equipment located at the at least one site location so as to change an operational parameter of the at least one environment-controlling equipment to mitigate the at least one verified environmental hazard type.
      2. The method according to clause 1, where the computing device may be located inside the at least one site location.
      3. The method according to clause 1, where the computing device may be located outside the at least one site location.
      4. The method according to clause 1, where the at least one site location may include a room;
where the at least one environment-controlling equipment may include a computer-controlled actuator; and
wherein the transmitting of the at least one second instruction may trigger the computer-controlled actuator to automatically: increase or decrease outdoor air fraction in the room, disperse indoor air in the room, raise or lower a temperature of the room, raise or lower humidity in the room, activate or deactivate air cleaners in the room, or any combination thereof.
   5. The method according to clause 1, where the at least one environment-controlling equipment may include at least one electronic circuitry in at least one communication device associated with at least one user; and
where the transmitting of the at least one second instruction may trigger the at least one electronic circuitry in the at least one communication device to alert the at least one user.
   6. The method according to clause 1, where the at least one site location may include at least one room in a building;
where the at least one environment-controlling equipment may include electronic circuitry in a central computing device in the building; and
where the transmitting of the at least one second instruction may trigger the electronic circuitry in the central computing device in the building to generate an audible alarm, a visual alert, an electronic alert message, or any combination thereof.
   7. The method according to clause 1, where the at least one site location may include at least one room in at least one building of an entity;
where the at least one environment-controlling equipment may include electronic circuitry in a central computing device at a predefined control-center location of the entity; and
where the transmitting of the at least one second instruction may trigger the electronic circuitry in the central computing device to generate an audible alarm, a visual alert, an electronic alert message, or any combination thereof.
   8. The method according to clause 1, where the at least one site location may include at least one room in a building and an outside of the building;
where the at least one environment-controlling equipment may include electronic circuitry in a central computing device in the building, and
further including determining, by the processor, that the overall health impact score outside of the building is greater than the overall health impact score inside the at least one room; and
causing, by the processor, over the communication network, to automatically open a window in the at least one room by the transmitting of the at least one second instruction to trigger a computer-controlled window actuator to automatically open the window.
   9. The method according to clause 1, where the health impact scoring algorithm may be based at least in part on using a value from each of the plurality of environmental output data measurements, a square of the value, and a cube of the value. (e.g., a polynomial equation of the value).
   10. The method according to clause 1, further including storing, by the processor, in a memory, the overall health impact score, the plurality of health impact scores, the at least one verified environmental hazard type, the at least one recommendation, or any combination thereof, that were measured at predefined time intervals.
   11. The method according to clause 10, where the transmitting of the first instructions may cause the display to display a history of the overall health impact score, the plurality of health impact scores, the at least one verified environmental hazard type, the at least one recommendation, or any combination thereof, that were measured at the predefined time intervals.
   12. The method according to clause 1, where the at least one particulate matter sensor may include a PM₁₀ particulate matter sensor, a PM_{2.5} particulate matter sensor, and a PM₁ particulate matter sensor that respectively output a PM_{2.5} particulate matter output data measurement, a PM_{2.5} particulate matter output data measurement, and a PM₁ particulate matter output data measurement; and where the executing an health impact scoring algorithm to compute the at least one particulate matter health impact score is based at least in part on the PM_{2.5} particulate matter output data measurement, the PM_{2.5} particulate matter output data measurement, and the PM₁ particulate matter output data measurement.
   13. A system may include a memory and a processor. The processor may be configured to execute computer code stored in the memory that causes the processor to:
receive over a communication network, from a plurality of environmental sensors, a plurality of environmental output data measurements of a plurality of environmental parameters;
   where the plurality of environmental sensors may be located inside of at least one site location, outside of the at least one site location, or both;
   where the plurality of environmental sensors may include:
   (i) an ozone sensor, outputting an ozone output data measurement of the plurality of environmental output data measurements,
   (ii) a humidity sensor, outputting a humidity output data measurement of the plurality of environmental output data measurements,
   (iii) a temperature sensor, outputting a temperature output data measurement of the plurality of environmental output data measurements,
   (iv) a carbon dioxide sensor, outputting a carbon dioxide output data measurement of the plurality of environmental output data measurements,
   (v) a carbon monoxide sensor, outputting a carbon monoxide output data measurement of the plurality of environmental output data measurements,
   (vi) a nitrous dioxide sensor, outputting a nitrous dioxide output data measurement of the plurality of environmental output data measurements,
   (vii) a sulfur dioxide sensor, outputting a sulfur dioxide output data measurement of the plurality of environmental output data measurements,
   (viii) a total volatile organic compound (tVOC) sensor, outputting a tVOC output data measurement of the plurality of environmental output data measurements, and
   (ix) at least one particulate matter sensor, outputting at least one particulate matter output data measurement of the plurality of environmental output data measurements;
execute a health impact scoring algorithm software to compute a plurality of health impact scores based at least in part on the plurality of environmental output data measurements from the plurality of environmental sensors;
   where a humidity health impact score of the plurality of health impact scores may be based at least in part on the humidity output data measurement;
   where an ozone health impact score of the plurality of health impact scores may be based at least in part on the ozone output data measurement;
   where a temperature health impact score of the plurality of health impact scores may be based at least in part on the temperature output data measurement;
   where a carbon dioxide health impact score of the plurality of health impact scores may be based at least in part on the carbon dioxide output data measurement;
   where a carbon monoxide health impact score of the plurality of health impact scores may be based at least in part on the carbon monoxide output data measurement;
   where a nitrous dioxide health impact score of the plurality of health impact scores may be based at least in part on the nitrous dioxide output data measurement;
   where a sulfur dioxide health impact score of the plurality of health impact scores may be based at least in part on the sulfur dioxide output data measurement;
   where a tVOC health impact score of the plurality of health impact scores may be based at least in part on the tVOC output data measurement;
   where at least one particulate matter health impact score of the plurality of health impact scores may be based at least in part on the at least one particulate matter output data measurement;
compute an overall health impact score in the at least one site location based at least in part on any of the plurality of health impact scores having a lowest health impact score;
determine at least one verified environmental hazard type in the at least one site location when at least one particular health impact score of the plurality of health impact scores is less than a respective predefined threshold score that is unique to the at least one particular environmental parameter;
generate by at least one machine learning model, at least one recommendation for remediating the at least one verified environmental hazard type when inputting the plurality of health impact scores into the at least one machine learning model;
transmit over the communication network, at least one of:
   (A) at least one first instruction to display on a computing device at least one of:
      (i) the overall health impact score,
      (ii) the at least one verified environmental hazard type, or
      (iii) the at least one recommendation; or
   (B) at least one second instruction to at least one environment-controlling equipment located at the at least one site location so as to change an operational parameter of the at least one environment-controlling equipment to mitigate the at least one verified environmental hazard type.
      14. The system according to clause 13, where the computing device may be located inside the at least one site location.
      15. The system according to clause 13, where the computing device may be located outside the at least one site location.
      16. The system according to clause 13, where the at least one site location may include a room;
where the at least one environment-controlling equipment may include a computer-controlled actuator; and
wherein the processor may be configured to transmit the at least one second instruction that triggers the computer-controlled actuator to automatically: increase or decrease outdoor air fraction in the room, disperse indoor air in the room, raise or lower a temperature of the room, raise or lower humidity in the room, activate or deactivate air cleaners in the room, or any combination thereof.
   17. The system according to clause 13, where the at least one environment-controlling equipment may include at least one electronic circuitry in at least one communication device associated with at least one user; and where the processor may be configured to transmit the at least one second instruction that triggers the at least one electronic circuitry in the at least one communication device to alert the at least one user.
   18. The system according to clause 13, where the at least one site location may include at least one room in a building;
where the at least one environment-controlling equipment may include electronic circuitry in a central computing device in the building; and
where the processor may be configured to transmit the at least one second instruction that triggers the electronic circuitry in the central computing device in the building to generate an audible alarm, a visual alert, an electronic alert message, or any combination thereof.
   19. The system according to clause 13, where the at least one site location may include at least one room in at least one building of an entity;
where the at least one environment-controlling equipment may include electronic circuitry in a central computing device at a predefined control-center location of the entity; and
where the processor may be configured to transmit the at least one second instruction that triggers the electronic circuitry in the central computing device to generate an audible alarm, a visual alert, an electronic alert message, or any combination thereof.
   20. The system according to clause 13, where the at least one site location may include at least one room in a building and an outside of the building;
where the at least one environment-controlling equipment may include electronic circuitry in a central computing device in the building, and
where the processor may be further configured to determine that the overall health impact score outside of the building is greater than the overall health impact score inside the at least one room, and
to cause over the communication network, to automatically open a window in the at least one room by the transmitting of the at least one second instruction to trigger a computer-controlled window actuator to automatically open the window.
   21. The system according to clause 13, where the health impact scoring algorithm may be based at least in part on using a value from each of the plurality of environmental output data measurements and a square of the value.
   22. The system according to clause 13, where the processor may be further configured to store in the memory: the overall health impact score, the plurality of health impact scores, the at least one verified environmental hazard type, the at least one recommendation, or any combination thereof, that were measured at predefined time intervals.
   23. The system according to clause 22, where the processor may be configured to transmit the first instructions that causes the display to display a history of the overall health impact score, the plurality of health impact scores, the at least one verified environmental hazard type, the at least one recommendation, or any combination thereof, that were measured at the predefined time intervals.

24. The system according to clause 13, where the at least one particulate matter sensor may include a PM₁₀ particulate matter sensor, a PM_{2.5} particulate matter sensor, and a PM₁ particulate matter sensor that respectively output a PM_{2.5} particulate matter output data measurement, a PM_{2.5} particulate matter output data measurement, and a PM₁ particulate matter output data measurement; and where the processor may be configured to execute an health impact scoring algorithm that computes the at least one particulate matter health impact score based at least in part on the PM_{2.5} particulate matter output data measurement, the PM_{2.5} particulate matter output data measurement, and the PM₁ particulate matter output data measurement.

25. A method may include:
continuously receiving, by a processor, over a communication network, from a plurality of environmental sensors, a plurality of environmental output data measurements of a plurality of environmental parameters;
   where the plurality of environmental parameters may include:
   (i) an ozone parameter,
   (ii) a humidity parameter,
   (iii) a temperature parameter,
   (iv) a carbon dioxide parameter,
   (v) a carbon monoxide parameter,
   (vi) a nitrous dioxide parameter,
   (vii) a sulfur dioxide parameter,
   (viii) a total volatile organic compound (tVOC) parameter, and
   (ix) a particulate matter parameter;
   where the plurality of environmental sensors may be located inside of a site location, outside of the site location, or both;
   where each particular sensor of the plurality of environmental sensors may be configured to continuously output a particular environmental output data measurement for a particular environmental parameter of the plurality of environmental parameters to obtain the plurality of environmental output data measurements;
automatically determining, by the processor, a particular health impact score for each particular environmental parameter of the plurality of environmental parameters based on the particular environmental output data measurement for the particular environmental parameter and at least one other environmental output data measurement from the plurality of environmental output data measurements to obtain a plurality of health impact scores associated with the plurality of environmental parameters;
   where each health impact score of the plurality of health impact scores may be associated with a particular health impact on a plurality of occupants in the site location;
defining, by the processor, an overall health impact score based on a lowest health impact score from the plurality of health impact scores;
receiving, by the processor, a personalized health data for each occupant from the plurality of occupants at the site location;
determining, by the processor:
   at least one verified environmental hazard type associated with at least one environmental parameter from the plurality of environmental parameters when at least one health impact score from the plurality of health impact scores is less than
   at least one predefined threshold score that is unique to the at least one environmental parameter, and
      at least one exposure time that the at least one verified environmental hazard type has been present at the site location;
utilizing, by the processor, at least one machine learning model to generate at least one personalized recommendation for reducing a health impact from the at least one verified environmental hazard type to each occupant based on inputting the plurality of health impact scores and the personalized health data for each occupant into the at least one machine learning model;
transmitting, by the processor, over the communication network, at least one first instruction to display on a computing device at least one of:
   (i) the overall health impact score,
   (ii) the at least one verified environmental hazard type,
   (iii) the at least one exposure time for the at least one verified environmental hazard type,
   (iv) the at least one personalized recommendation, or
   (v) at least one possible health effect on each occupant based on the at least one exposure time to the at least one verified environmental hazard type; and
automatically transmitting, by the processor, over the communication network, at least one second instruction to at least one environment-controlling equipment associated with the site location to change an operational parameter of the at least one environment-controlling equipment to mitigate the at least one verified environmental hazard type.
   26. The method according to clause 25, may further include determining, by the processor, using location tracking data and the at least one exposure time that the at least one verified environmental hazard type was present in the site location, at least one personal exposure time that each occupant has been exposed to the at least one verified environmental hazard type in the site location.
   27. The method according to clause 25, may further include generating, by the processor, a personalized report for each occupant at the site location;
where the personalized report may include:
   the at least one personal exposure time that each occupant was exposed to the at least one verified environmental hazard type, and
   at least one possible health effect on each occupant based on the at least one personal exposure time that each occupant has been exposed to the at least one verified environmental hazard type.
      28. The method according to clause 26, may further include transmitting, by the processor, at least one third instruction to a computing device to display a personalized alert associated with each occupant at the site location;
where the personalized alert may include:
   a notification of a presence of the at least one verified environmental hazard type in the site location, and
   the at least one personalized recommendation for reducing an exposure to the at least one verified environmental hazard type in the site location.
      29. The method according to clause 28, where the computing device associated with each occupant is a smartphone, a wearable device, or both;
where the receiving may include receiving the personalized health data for each occupant that is stored on the smartphone, the wearable device, or both associated with each occupant;
where the transmitting of the at least one third instruction to display the personalized alert on the smartphone, the wearable device, or both associated with each occupant from the plurality of occupants.
   30. The method according to clause 25, where the site location may include a room;
where the at least one environment-controlling equipment may include a computer-controlled actuator; and
where the transmitting of the at least one second instruction may trigger the computer-controlled actuator to automatically: increase or decrease outdoor air fraction in the room, disperse indoor air in the room, raise or lower a temperature of the room, raise or lower humidity in the room, activate or deactivate air cleaners in the room, or any combination thereof.
   31. The method according to clause 25, where the at least one environment-controlling equipment may include at least one electronic circuitry in at least one communication device associated with at least one user; and
where the transmitting of the at least one second instruction may trigger the at least one electronic circuitry in the at least one communication device to alert the at least one user.
   32. The method according to clause 25, where the at least one environment-controlling equipment may include electronic circuitry in a central computing device in the site location; and where the transmitting of the at least one second instruction may trigger the electronic circuitry in the central computing device in the site location to generate an audible alarm, a visual alert, an electronic alert message, or any combination thereof.
   33. The method according to clause 25, where the at least one environment-controlling equipment may include electronic circuitry in a central computing device at a predefined control-center location of an entity; and
where the transmitting of the at least one second instruction may trigger the electronic circuitry in the central computing device to generate an audible alarm, a visual alert, an electronic alert message, or any combination thereof.
   34. The method according to clause 25, where the site location may include at least one room;
where the at least one environment-controlling equipment may include electronic circuitry in a central computing device in the site location, and
may further include determining, by the processor, that the overall health impact score outside of the site location is greater than the overall health impact score inside the at least one room; and
causing, by the processor, over the communication network, to automatically open a window in the at least one room by the transmitting of the at least one second instruction to trigger a computer-controlled window actuator to automatically open the window.
   35. The method according to clause 25, may include storing, by the processor, in a memory, each particular overall health impact score, each particular verified environmental hazard type, each particular recommendation, or any combination thereof, that were measured at predefined time intervals.
   36. The method according to clause 35, where the transmitting of the at least one first instruction may cause the computing device to display a history of: each particular overall health impact score, each particular verified environmental hazard type, each particular recommendation, or any combination thereof, that were measured at the predefined time intervals.

37. The method according to clause 25, where the particulate matter parameter may include a PM₁₀ particulate matter parameter, a PM_{2.5} particulate matter parameter, and a PM₁ particulate matter parameter and where the automatic determining of the particular health impact may be based at least in part on a PM₁₀ particulate matter output data measurement for the PM₁₀ particulate matter parameter, a PM_{2.5} particulate matter output data measurement for the PM_{2.5} particulate matter parameter, and a PM₁ particulate matter output data measurement for the PM₁ particulate matter parameter.

38. A system may include:
a memory; and
a processor configured to execute computer code stored in the memory that causes the processor to:
continuously receive over a communication network, from a plurality of environmental sensors, a plurality of environmental output data measurements of a plurality of environmental parameters;
where the plurality of environmental parameters may include:
   (i) an ozone parameter,
   (ii) a humidity parameter,
   (iii) a temperature parameter,
   (iv) a carbon dioxide parameter,
   (v) a carbon monoxide parameter,
   (vi) a nitrous dioxide parameter,
   (vii) a sulfur dioxide parameter,
   (viii) a total volatile organic compound (tVOC) parameter, and
   (ix) a particulate matter parameter;
   where the plurality of environmental sensors may be located inside of a site location, outside of the site location, or both;
   where each particular sensor of the plurality of environmental sensors may be configured to continuously output a particular environmental output data measurement for a particular environmental parameter of the plurality of environmental parameters to obtain a plurality of environmental output data measurements;
automatically determine a particular health impact score for each particular environmental parameter of the plurality of environmental parameters based on the particular environmental output data measurement for the particular environmental parameter and at least one other environmental output data measurement from the plurality of environmental output data measurements to obtain a plurality of health impact scores associated with the plurality of environmental parameters;
   where each health impact score of the plurality of health impact scores may be associated with a particular health impact on a plurality of occupants in the site location;
define an overall health impact score based on a lowest health impact score from the plurality of health impact scores;
receive a personalized health data for each occupant from the plurality of occupants at the site location;
determine:
   at least one verified environmental hazard type associated with at least one environmental parameter from the plurality of environmental parameters when at least one health impact score from the plurality of health impact scores is less than at least one predefined threshold score that is unique to the at least one environmental parameter, and
   at least one exposure time that the at least one verified environmental hazard type has been present at the site location;
utilize at least one machine learning model to generate at least one personalized recommendation for reducing a health impact from the at least one verified environmental hazard type to each occupant based on inputting the plurality of health impact scores and the personalized health data for each occupant into the at least one machine learning model;
transmit over the communication network, at least one first instruction to display on a computing device at least one of:
   (i) the overall health impact score,
   (ii) the at least one verified environmental hazard type,
   (iii) the at least one exposure time for the at least one verified environmental hazard type,
   (iv) the at least one personalized recommendation, or
   (v) at least one possible health effect on each occupant based on the at least one exposure time to the at least one verified environmental hazard type; and
automatically transmit over the communication network, at least one second instruction to at least one environment-controlling equipment associated with the site location to change an operational parameter of the at least one environment-controlling equipment to mitigate the at least one verified environmental hazard type.
   39. The system according to clause 38, where the processor may be further configured to determine, using location tracking data and the at least one exposure time that the at least one verified environmental hazard type was present in the site location, at least one personal exposure time that each occupant has been exposed to the at least one verified environmental hazard type in the site location.
   40. The system according to clause 39, where the processor may be further configured to generate a personalized report for each occupant at the site location;
where the personalized report may include:
   the at least one personal exposure time that each occupant was exposed to the at least one verified environmental hazard type, and
   at least one possible health effect on each occupant based on the at least one personal exposure time that each occupant has been exposed to the at least one verified environmental hazard type.
      41. The system according to clause 40, where the processor is configured to transmit at least one third instruction to a computing device to display a personalized alert associated with each occupant at the site location;
where the personalized alert may include:
   a notification of a presence of the at least one verified environmental hazard type in the site location, and
   the at least one personalized recommendation for reducing an exposure to the at least one verified environmental hazard type in the site location.
      42. The system according to clause 41, where the computing device associated with each occupant may be a smartphone, a wearable device, or both;
where the processor may be configured to receive the personalized health data for each occupant that is stored on the smartphone, the wearable device, or both associated with each occupant; and to transmit the at least one third instruction to display the personalized alert on the smartphone, the wearable device, or both associated with each occupant from the plurality of occupants.
   43. The system according to clause 38, where the site location may include a room;
where the at least one environment-controlling equipment may include a computer-controlled actuator; and
where the processor may be configured to transmit the at least one second instruction that triggers the computer-controlled actuator to automatically: increase or decrease outdoor air fraction in the room, disperse indoor air in the room, raise or lower a temperature of the room, raise or lower humidity in the room, activate or deactivate air cleaners in the room, or any combination thereof.
   44. The system according to clause 38, where the at least one environment-controlling equipment may include at least one electronic circuitry in at least one communication device associated with at least one user; and
where the processor may be configured to transmit the at least one second instruction that triggers the at least one electronic circuitry in the at least one communication device to alert the at least one user.
   45. The system according to clause 38,
where the at least one environment-controlling equipment may include electronic circuitry in a central computing device in the site location; and
where the processor may be configured to transmit the at least one second instruction that triggers the electronic circuitry in the central computing device in the site location to generate an audible alarm, a visual alert, an electronic alert message, or any combination thereof.
   46. The system according to clause 38, where the at least one environment-controlling equipment may include electronic circuitry in a central computing device at a predefined control-center location of an entity; and
where the processor may be configured to transmit the at least one second instruction that triggers the electronic circuitry in the central computing device to generate an audible alarm, a visual alert, an electronic alert message, or any combination thereof.
   47. The system according to clause 38, where the site location may include at least one room;
where the at least one environment-controlling equipment may include electronic circuitry in a central computing device in the site location, and
where the processor may be further configured to determine that the overall health impact score outside of the site location is greater than the overall health impact score inside the at least one room, and to cause over the communication network, to automatically open a window in the at least one room by the transmitting of the at least one second instruction to trigger a computer-controlled window actuator to automatically open the window.
   48. The system according to clause 38, where the processor may be further configured to store in the memory: each particular overall health impact score, each particular verified environmental hazard type, each particular recommendation, or any combination thereof, that were measured at predefined time intervals.
   49. The system according to clause 48, where the processor may be configured to transmit the at least one first instruction that causes the computing device to display a history of: each particular overall health impact score, each particular verified environmental hazard type, each particular recommendation, or any combination thereof, that were measured at the predefined time intervals.
   50. The system according to clause 38, where the particulate matter parameter may include a PM₁₀ particulate matter parameter, a PM_{2.5} particulate matter parameter, and a PM₁ particulate matter parameter and where the processor may be configured to automatically determine the particular a health impact score based at least in part on a PM₁₀ particulate matter output data measurement for the PM₁₀ particulate matter parameter, a PM_{2.5} particulate matter output data measurement for the PM_{2.5} particulate matter parameter, and a PM₁ particulate matter output data measurement for the PM₁ particulate matter parameter.
   51. A method may include:
continuously receiving, by a processor, over a communication network, from a plurality of environmental sensors, a plurality of environmental output data measurements of a plurality of environmental parameters;
where the plurality of environmental parameters may include at least three of:
   (i) an ozone parameter,
   (ii) a humidity parameter,
   (iii) a temperature parameter,
   (iv) a carbon dioxide parameter,
   (v) a carbon monoxide parameter,
   (vi) a nitrous dioxide parameter,
   (vii) a sulfur dioxide parameter,
   (viii) a total volatile organic compound (tVOC) parameter,
   (ix) a particulate matter parameter; and
   (x) any combination thereof;
where the plurality of environmental sensors may be located inside of a building, outside of the building, or both;
determining, by the processor, at least one health impact score based on the plurality of environmental output data measurements of at least two of the plurality of environmental parameters;
where the at least one health impact score may be associated with a particular health impact on at least one occupant in the building;
automatically optimizing, by the processor, an indoor environment for a health of at least one occupant of the building, by:
determining, by the processor, the at least one health impact score is less than a predefined threshold score;
determining, by the processor, at least one verified environmental hazard type associated with the at least one health impact score being less than the predefined threshold score;
   inputting, by the processor, the at least one health impact score into at least one algorithm to generate at least one recommendation for remediating the at least one verified environmental hazard type;
transmitting, by the processor, over the communication network, at least one of:
(A) at least one first instruction to display on a computing device at least one of:
   (i) the at least one health impact score,
   (ii) the at least one verified environmental hazard type, or
   (iii) the at least one recommendation; or
(B) at least one second instruction to at least one environment-controlling equipment associated with the building so as to change an operational parameter of the at least one environment-controlling equipment to mitigate each particular verified environmental hazard type; and
confirming, by the processor, the at least one health impact score is equal to or above the predefined threshold score after the at least one recommendation has been implemented.
   52. The method according to clause 51, where the at least one algorithm may include at least one machine learning model.
   53. The method according to clause 51, where the receiving of the plurality of environmental output data measurements further includes receiving at least one environmental output data measurement from a third-party database.
   54. The method according to clause 51,
where the plurality of environmental parameters may include the ozone parameter, the humidity parameter, and at least one of:
   (i) the temperature parameter,
   (ii) the carbon dioxide parameter,
   (iii) the carbon monoxide parameter,
   (iv) the nitrous dioxide parameter,
   (v) the sulfur dioxide parameter,
   (vi) the total volatile organic compound (tVOC) parameter,
   (vii) the particulate matter parameter; and
   (viii) any combination thereof; and
where the determining of the at least one health impact score may be based on:
at least one first environmental output data measurement of the ozone parameter,
at least one second environmental output data measurement of the humidity parameter, and
at least one third environmental output data measurement of at least one of:
   (i) the temperature parameter,
   (ii) the carbon dioxide parameter,
   (iii) the carbon monoxide parameter,
   (iv) the nitrous dioxide parameter,
   (v) the sulfur dioxide parameter,
   (vi) the total volatile organic compound (tVOC) parameter,
   (vii) the particulate matter parameter; and
   (viii) any combination thereof.
      55. The method according to clause 54, where the at least one algorithm may include at least one machine learning model.
      56. The method according to clause 54, where the receiving of the plurality of environmental output data measurements receiving at least one environmental output data measurement from a third-party database.
      57. A method may include:
continuously receiving, by a processor, over a communication network, from a plurality of environmental sensors, a plurality of environmental output data measurements of a plurality of environmental parameters;
   where the plurality of environmental parameters may include:
      an ozone parameter,
      a humidity parameter,
      a temperature parameter,
      a carbon dioxide parameter,
      a carbon monoxide parameter,
      a nitrous dioxide parameter,
      a sulfur dioxide parameter,
      a total volatile organic compound (tVOC) parameter, and
      a particulate matter parameter;
   where the plurality of environmental sensors may be located inside of a site location, outside of the site location, or both;
   where each particular sensor of the plurality of environmental sensors may be configured to continuously output a particular environmental output data measurement for a particular environmental parameter of the plurality of environmental parameters to obtain a plurality of environmental output data measurements;
automatically determining, by the processor, based on the plurality of environmental output data measurements, at least one human health hazard associated with at least one environmental parameter from the plurality of environmental parameters for at least one occupant in an indoor environment at the site location;
utilizing, by the processor, at least one machine learning model to generate at least one recommendation to facilitate a change in the at least one environmental parameter in the indoor environment to remediate the at least one human health hazard; and
automatically transmitting, by the processor, over the communication network, at least one instruction to at least one environment-controlling equipment associated with the site location to change an operational parameter of the at least one environment-controlling equipment to facilitate the change in the at least one environmental parameter in the indoor environment to mitigate the at least one human health hazard based on the at least one recommendation.
   58. The method according to clause 1, where the at least one human health hazard is at least one of:
an immune system disruption,
a respiratory disease,
an organ dysfunction,
a microbiome disruption,
cell damage, or
tissue damage.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Further still, the various steps may be carried out in any desired order (and any desired steps may be added and/or any desired steps may be eliminated).

1. A method, comprising:
continuously receiving, by a processor, over a communication network, from a plurality of environmental sensors, a plurality of environmental output data measurements of a plurality of environmental parameters;
wherein the plurality of environmental parameters comprises:
(i) an ozone parameter,
(ii) a humidity parameter,
(iii) a temperature parameter,
(iv) a carbon dioxide parameter,
(v) a carbon monoxide parameter,
(vi) a nitrous dioxide parameter,
(vii) a sulfur dioxide parameter,
(viii) a total volatile organic compound (tVOC) parameter, and
(ix) a particulate matter parameter;
wherein the plurality of environmental sensors is located inside of a site location, outside of the site location, or both;
wherein each particular sensor of the plurality of environmental sensors is configured to continuously output a particular environmental output data measurement for a particular environmental parameter of the plurality of environmental parameters to obtain the plurality of environmental output data measurements;
automatically determining, by the processor, a particular health impact score for each particular environmental parameter of the plurality of environmental parameters based on the particular environmental output data measurement for the particular environmental parameter and at least one other environmental output data measurement from the plurality of environmental output data measurements to obtain a plurality of health impact scores associated with the plurality of environmental parameters;
wherein each health impact score of the plurality of health impact scores is associated with a particular health impact on a plurality of occupants in the site location;
defining, by the processor, an overall health impact score based on a lowest health impact score from the plurality of health impact scores;
receiving, by the processor, a personalized health data for each occupant from the plurality of occupants at the site location;
determining, by the processor:
at least one verified environmental hazard type associated with at least one environmental parameter from the plurality of environmental parameters when at least one health impact score from the plurality of health impact scores is less than at least one predefined threshold score that is unique to the at least one environmental parameter, and
at least one exposure time that the at least one verified environmental hazard type has been present at the site location;
utilizing, by the processor, at least one machine learning model to generate at least one personalized recommendation for reducing a health impact from the at least one verified environmental hazard type to each occupant based on inputting the plurality of health impact scores and the personalized health data for each occupant into the at least one machine learning model;
transmitting, by the processor, over the communication network, at least one first instruction to display on a computing device at least one of:
(i) the overall health impact score,
(ii) the at least one verified environmental hazard type,
(iii) the at least one exposure time for the at least one verified environmental hazard type,
(iv) the at least one personalized recommendation, or
(v) at least one possible health effect on each occupant based on the at least one exposure time to the at least one verified environmental hazard type; and
automatically transmitting, by the processor, over the communication network, at least one second instruction to at least one environment-controlling equipment associated with the site location to change an operational parameter of the at least one environment-controlling equipment to mitigate the at least one verified environmental hazard type.

2. The method according to claim 1, further comprising determining, by the processor, using location tracking data and the at least one exposure time that the at least one verified environmental hazard type was present in the site location, at least one personal exposure time that each occupant has been exposed to the at least one verified environmental hazard type in the site location.

3. The method according to claim 2, further comprising generating, by the processor, a personalized report for each occupant at the site location;
wherein the personalized report comprises:
the at least one personal exposure time that each occupant was exposed to the at least one verified environmental hazard type, and
at least one possible health effect on each occupant based on the at least one personal exposure time that each occupant has been exposed to the at least one verified environmental hazard type.

4. The method according to claim 2, further comprising transmitting, by the processor, at least one third instruction to a computing device to display a personalized alert associated with each occupant at the site location;
wherein the personalized alert comprises:
a notification of a presence of the at least one verified environmental hazard type in the site location, and
the at least one personalized recommendation for reducing an exposure to the at least one verified environmental hazard type in the site location.

5. The method according to claim 4, wherein the computing device associated with each occupant is a smartphone, a wearable device, or both;
wherein the receiving comprises receiving the personalized health data for each occupant that is stored on the smartphone, the wearable device, or both associated with each occupant;
wherein the transmitting of the at least one third instruction to display the personalized alert on the smartphone, the wearable device, or both associated with each occupant from the plurality of occupants.

6. The method according to claim 1, wherein the site location comprises a room;
wherein the at least one environment-controlling equipment comprises a computer-controlled actuator; and
wherein the transmitting of the at least one second instruction triggers the computer-controlled actuator to automatically: increase or decrease outdoor air fraction in the room, disperse indoor air in the room, raise or lower a temperature of the room, raise or lower humidity in the room, activate or deactivate air cleaners in the room, or any combination thereof.

7. The method according to claim 1, wherein the at least one environment-controlling equipment comprises at least one electronic circuitry in at least one communication device associated with at least one user; and
wherein the transmitting of the at least one second instruction triggers the at least one electronic circuitry in the at least one communication device to alert the at least one user.

8. The method according to claim 1, wherein the at least one environment-controlling equipment comprises electronic circuitry in a central computing device in the site location; and
wherein the transmitting of the at least one second instruction triggers the electronic circuitry in the central computing device in the site location to generate an audible alarm, a visual alert, an electronic alert message, or any combination thereof.

9. The method according to claim 1,
wherein the at least one environment-controlling equipment comprises electronic circuitry in a central computing device at a predefined control-center location of an entity; and
wherein the transmitting of the at least one second instruction triggers the electronic circuitry in the central computing device to generate an audible alarm, a visual alert, an electronic alert message, or any combination thereof.

10. The method according to claim 1, wherein the site location comprises at least one room;
wherein the at least one environment-controlling equipment comprises electronic circuitry in a central computing device in the site location, and
further comprising determining, by the processor, that the overall health impact score outside of the site location is greater than the overall health impact score inside the at least one room; and
causing, by the processor, over the communication network, to automatically open a window in the at least one room by the transmitting of the at least one second instruction to trigger a computer-controlled window actuator to automatically open the window.

11. The method according to claim 1, further comprising storing, by the processor, in a memory, each particular overall health impact score, each particular verified environmental hazard type, each particular recommendation, or any combination thereof, that were measured at predefined time intervals.

12. The method according to claim 11, wherein the transmitting of the at least one first instruction causes the computing device to display a history of: each particular overall health impact score, each particular verified environmental hazard type, each particular recommendation, or any combination thereof, that were measured at the predefined time intervals.

13. The method according to claim 1, wherein the particulate matter parameter comprises a PM₁₀ particulate matter parameter, a PM_{2.5} particulate matter parameter, and a PM₁ particulate matter parameter and wherein the automatic determining of the particular health impact is based at least in part on a PM₁₀ particulate matter output data measurement for the PM₁₀ particulate matter parameter, a PM_{2.5} particulate matter output data measurement for the PM_{2.5} particulate matter parameter, and a PM₁ particulate matter output data measurement for the PM₁ particulate matter parameter.

14. A system, comprising:
a memory; and
a processor configured to execute computer code stored in the memory that causes the processor to:
continuously receive over a communication network, from a plurality of environmental sensors, a plurality of environmental output data measurements of a plurality of environmental parameters;
wherein the plurality of environmental parameters comprises:
(i) an ozone parameter,
(ii) a humidity parameter,
(iii) a temperature parameter,
(iv) a carbon dioxide parameter,
(v) a carbon monoxide parameter,
(vi) a nitrous dioxide parameter,
(vii) a sulfur dioxide parameter,
(viii) a total volatile organic compound (tVOC) parameter, and
(ix) a particulate matter parameter;
wherein the plurality of environmental sensors is located inside of a site location, outside of the site location, or both;
wherein each particular sensor of the plurality of environmental sensors is configured to continuously output a particular environmental output data measurement for a particular environmental parameter of the plurality of environmental parameters to obtain a plurality of environmental output data measurements;
automatically determine a particular health impact score for each particular environmental parameter of the plurality of environmental parameters based on the particular environmental output data measurement for the particular environmental parameter and at least one other environmental output data measurement from the plurality of environmental output data measurements to obtain a plurality of health impact scores associated with the plurality of environmental parameters;
wherein each health impact score of the plurality of health impact scores is associated with a particular health impact on a plurality of occupants in the site location;
define an overall health impact score based on a lowest health impact score from the plurality of health impact scores;
receive a personalized health data for each occupant from the plurality of occupants at the site location;
determine:
at least one verified environmental hazard type associated with at least one environmental parameter from the plurality of environmental parameters when at least one health impact score from the plurality of health impact scores is less than at least one predefined threshold score that is unique to the at least one environmental parameter, and
at least one exposure time that the at least one verified environmental hazard type has been present at the site location;
utilize at least one machine learning model to generate at least one personalized recommendation for reducing a health impact from the at least one verified environmental hazard type to each occupant based on inputting the plurality of health impact scores and the personalized health data for each occupant into the at least one machine learning model;
transmit over the communication network, at least one first instruction to display on a computing device at least one of:
(i) the overall health impact score,
(ii) the at least one verified environmental hazard type,
(iii) the at least one exposure time for the at least one verified environmental hazard type,
(iv) the at least one personalized recommendation, or
(v) at least one possible health effect on each occupant based on the at least one exposure time to the at least one verified environmental hazard type; and
automatically transmit over the communication network, at least one second instruction to at least one environment-controlling equipment associated with the site location to change an operational parameter of the at least one environment-controlling equipment to mitigate the at least one verified environmental hazard type.

15. The system according to claim 14, wherein the processor is further configured to determine, using location tracking data and the at least one exposure time that the at least one verified environmental hazard type was present in the site location, at least one personal exposure time that each occupant has been exposed to the at least one verified environmental hazard type in the site location.

16. The system according to claim 15, wherein the processor is further configured to generate a personalized report for each occupant at the site location;
wherein the personalized report comprises:
the at least one personal exposure time that each occupant was exposed to the at least one verified environmental hazard type, and
at least one possible health effect on each occupant based on the at least one personal exposure time that each occupant has been exposed to the at least one verified environmental hazard type.

17. The system according to claim 16, wherein the processor is configured to transmit at least one third instruction to a computing device to display a personalized alert associated with each occupant at the site location;
wherein the personalized alert comprises:
a notification of a presence of the at least one verified environmental hazard type in the site location, and
the at least one personalized recommendation for reducing an exposure to the at least one verified environmental hazard type in the site location.

18. The system according to claim 17, wherein the computing device associated with each occupant is a smartphone, a wearable device, or both;
wherein the processor is configured to receive the personalized health data for each occupant that is stored on the smartphone, the wearable device, or both associated with each occupant; and to transmit the at least one third instruction to display the personalized alert on the smartphone, the wearable device, or both associated with each occupant from the plurality of occupants.

19. The system according to claim 14, wherein the site location comprises a room;
wherein the at least one environment-controlling equipment comprises a computer-controlled actuator; and
wherein the processor is configured to transmit the at least one second instruction that triggers the computer-controlled actuator to automatically: increase or decrease outdoor air fraction in the room, disperse indoor air in the room, raise or lower a temperature of the room, raise or lower humidity in the room, activate or deactivate air cleaners in the room, or any combination thereof.

20. The system according to claim 14, wherein the at least one environment-controlling equipment comprises at least one electronic circuitry in at least one communication device associated with at least one user; and
wherein the processor is configured to transmit the at least one second instruction that triggers the at least one electronic circuitry in the at least one communication device to alert the at least one user.

21. The system according to claim 14,
wherein the at least one environment-controlling equipment comprises electronic circuitry in a central computing device in the site location; and
wherein the processor is configured to transmit the at least one second instruction that triggers the electronic circuitry in the central computing device in the site location to generate an audible alarm, a visual alert, an electronic alert message, or any combination thereof.

22. The system according to claim 14,
wherein the at least one environment-controlling equipment comprises electronic circuitry in a central computing device at a predefined control-center location of an entity; and
wherein the processor is configured to transmit the at least one second instruction that triggers the electronic circuitry in the central computing device to generate an audible alarm, a visual alert, an electronic alert message, or any combination thereof.

23. The system according to claim 14, wherein the site location comprises at least one room;
wherein the at least one environment-controlling equipment comprises electronic circuitry in a central computing device in the site location, and
wherein the processor is further configured to determine that the overall health impact score outside of the site location is greater than the overall health impact score inside the at least one room, and to cause over the communication network, to automatically open a window in the at least one room by the transmitting of the at least one second instruction to trigger a computer-controlled window actuator to automatically open the window.

24. The system according to claim 14, wherein the processor is further configured to store in the memory: each particular overall health impact score, each particular verified environmental hazard type, each particular recommendation, or any combination thereof, that were measured at predefined time intervals.

25. The system according to claim 24, wherein the processor is configured to transmit the at least one first instruction that causes the computing device to display a history of: each particular overall health impact score, each particular verified environmental hazard type, each particular recommendation, or any combination thereof, that were measured at the predefined time intervals.

26. The system according to claim 14, wherein the particulate matter parameter comprises a PM₁₀ particulate matter parameter, a PM_{2.5} particulate matter parameter, and a PM₁ particulate matter parameter and wherein the processor is configured to automatically determine the particular health impact score based at least in part on a PM₁₀ particulate matter output data measurement for the PM₁₀ particulate matter parameter, a PM_{2.5} particulate matter output data measurement for the PM_{2.5} particulate matter parameter, and a PM₁ particulate matter output data measurement for the PM₁ particulate matter parameter.
